(19) United States
(12) Patent Application Publication
(10) Pub. No.: US 2018/0015074 A1
(43) Pub. Date: Jan. 18, 2018

(54) USE OF MTOR INHIBITORS TO PREVENT AND REGRESS EDHESIONS AND FIBROSIS

(71) Applicant: THE BOARD OF REGENTS OF THE UNIVERSITY OF TEXAS SYSTEM, Austin, TX (US)

(72) Inventor: Dean L. KELLOGG, San Antonio, TX (US)

(21) Appl. No.: 15/548,357

(22) PCT Filed: Feb. 8, 2016
(86) PCT No.: PCT/US16/17022

§ 371 Date: Aug. 2, 2017

Related U.S. Application Data
(60) Provisional application No. 62/113,667, filed on Feb. 9, 2015.

(51) Int. Cl. A61K 31/436; A61K 9/46; A61K 9/00; A61K 9/50

(52) U.S. Cl. CPC A61K 31/436; A61K 9/5073; A61K 9/5047; A61K 9/5042; A61K 9/0007; A61K 9/5031; A61K 9/5026; A61K 9/0014; A61K 9/5036

(57) ABSTRACT

Embodiments of the disclosure include preventing or reducing adhesion between two tissues and/or organs in an individual subjected to a procedure and/or preventing or reducing one or more keloids in an individual subjected to a procedure by providing to the individual an effective amount of a composition comprising one or more inhibitors of an mTOR pathway no earlier than about 4 days following the procedure.

This application claims priority to U.S. Provisional Patent Application Ser. No. 62/113,667, filed Feb. 9, 2015, which is incorporated by reference herein in its entirety.

### STATEMENT REGARDING FEDERALLY SPONSORED RESEARCH OR DEVELOPMENT

This invention was made with government support under R21AG041365-01A1 awarded by the National Institute on Aging. The government has certain rights in the invention.

### TECHNICAL FIELD

Embodiments of the disclosure include at least the fields of cell biology, molecular biology, and medicine, including surgical medicine.

### BACKGROUND

Adhesions are fibrous bands of internal scar tissue that form between tissues and organs, typically following abdominal or gynecological surgeries. Adhesions develop after nearly every abdominal surgery, they begin forming within hours and within a few weeks cause internal organs to attach to the surgical site or to other organs in the abdominal cavity, frequently resulting in abdominal pain or intestinal obstruction.

Common adhesion-related complications include small bowel obstruction, female infertility, and chronic pelvic pain. In patients who have abdominal surgery, 93% will develop adhesions that may require a second operation to break the adhesions. Nearly 350,000 procedures are performed yearly to lyse peritoneal adhesions. Even after adhesion lysis, recurrent obstruction and re-operation is common, further adding to the physical, emotional, and financial costs.

Adhesions form due to excess production and deposition of extracellular matrix, especially collagen and fibronectin. Compared to normal tissue, adhesions express a number of genes that regulate cell growth and apoptosis, inflammation, angiogenesis, and tissue turnover, many of which are under the control of the mTOR pathway. Inhibition of the mTOR pathway by rapamycin leads to an increase in collagenase, which could potentially break down excess collagen deposition in adhesions.

Described herein is a solution to a long-felt need in the art to provide therapy for the prevention or treatment of adhesions.

### BRIEF SUMMARY

Embodiments of the disclosure encompass administration of one or more mTOR inhibitors (such as rapamycin and/or rapamycin analogs) in an individual following a medical procedure to reduce or prevent adhesion formation, keloid formation, and/or fibrosis. Although the medical procedure can be of any kind, in specific embodiments the adhesion(s) are or could be between abdominal, pelvic, or thoracic organs and/or with the walls of the abdominal, pelvic, or thoracic cavities, for example.

In particular embodiments, methods of the disclosure encompass preventing or reducing adhesion between two tissues and/or organs in an individual subjected to a medical procedure. In some embodiments, methods of the disclosure include preventing or reducing one or more keloids in an individual subjected to a procedure. In certain embodiments, methods of the disclosure concern preventing or reducing fibrosis in an individual following a procedure.

In embodiments of the disclosure, rapamycin and/or a rapamycin analog (or any mTOR inhibitor) is provided to an individual after a procedure but no earlier than a certain amount of time after the procedure. In specific embodiments, a particular amount of time must pass before the rapamycin and/or rapamycin analog is given to the individual. In particular embodiments, a window of time must occur before the rapamyin and/or rapamycin analog is given to the individual in order to allow sufficient wound healing to begin or occur. In at least certain cases, if a rapamycin and/or rapamycin analog is given before a specific amount of time has occurred since the procedure, the wound from the procedure will not heal sufficiently or properly.

In particular embodiments, the rapamycin and/or rapamycin analog (or any mTOR inhibitor) must not be provided to the individual any earlier than 2 days, 3 days, 4 days, 5 days, or 6 days or more, and so on, following the procedure. In specific embodiments, the rapamycin and/or rapamycin analog must not be provided to the individual any earlier than about 24, 25, 26, 27,28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, or more hours following the procedure.

An effective amount of rapamycin and/or rapamycin analog (or any mTOR inhibitor) or derivative will depend upon the adhesion(s) or keloid(s) to be treated, the length of duration desired and the bioavailability profile of the composition, and the site of administration. In some embodiments, the composition comprises rapamycin and/or an analog thereof at a concentration of 0.001 mg to 30 mg total per dose. In some embodiments, the composition comprising rapamycin or an analog of rapamycin comprises 0.001% to 60% by weight of rapamycin or an analog of rapamycin. In some embodiments, the average blood level of rapamycin in the subject is greater than 0.5 ng per mL whole blood after administration of the composition.

The composition can be administered to the subject using any method known to those of ordinary skill in the art. In some embodiments, the composition may be administered intravenously, intracerebrally, intracranially, intraventricularly, intrathecally, into the cortex, thalamus, hypothalamus, hippocampus, basal ganglia, substantia nigra or the region of the substantia nigra, cerebellum, intradermally, intraarterially, intraperitoneally, intralesionally, intratracheally, intranasally, topically, intramuscularly, intraperitoneally, anally, subcutaneously, orally, topically, locally, inhalation (e.g., aerosol inhalation), injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art. In some embodiments, the composition is administered orally, enterically, colonically, anally, intravenously, or dermally with a patch. In some embodiments, the composition comprising rapamycin or an analog of rapamycin is comprised in a food or food additive.

The dose can be repeated as needed as determined by those of ordinary skill in the art so long as the initial dose occurs after sufficient time is given for the wound to begin healing and/or after sufficient time that the rapamayin and/or rapamycin analog does not interfere with healing. In some embodiments, the rapamycin or analog of rapamycin is administered in two or more doses. Where more than one dose is administered to a subject, the time interval between doses can be any time interval as determined by those of ordinary skill in the art. For example, the two doses may be 1, 2, 3, 4, 5, 6, 7, 8, 9, 10, 11, 12, 13, 14, 15, 16, 17, 18, 29, 20, 21, 22, 23, or 24 hours apart, or any range therein. In some embodiments, the composition may be administered daily, weekly, monthly, annually, or any range therein. In some embodiments, the interval of time between administration of doses comprising rapamycin or an analog of rapamycin is between 0.5 to 30 days, 1 to 30 days, 1 to 21 days, 1 to 14 days, 7 to 30 days, 7 to 21 days, 7 to 14 days, 14 to 30 days, 14 to 21 days, or 21 to 30 days, for example.

In some embodiments, the method comprises further administering one or more secondary or additional forms of therapies. In some embodiments, the subject is further administered a composition comprising a second active agent. In specific embodiments, a second therapy is utilized, such as surgery, occlusive dressings, compression therapy, intralesional corticosteroid injections, cryosurgery, excision, radiation therapy, laser therapy, interferon therapy, 5-fluorouracil, doxorubicin, bleomycin, verapamil, retinoic acid, a combination thereof, and so forth. In some embodiments, the composition comprising rapamycin or an analog of rapamycin is administered at the same time as the composition comprising the second active agent. In some embodiments, the composition comprising rapamycin and/or an analog of rapamycin is administered before or after the composition comprising the second active agent is administered. In some embodiments, the two treatments may be 1, 2, 3, 4, 5, 6, 7, 8, 9, 10, 11, 12, 13, 14, 15, 16, 17, 18, 29, 20, 21, 22, 23, or 24 hours apart, 1, 2, 3, 4, 5, 6, 7, 8, 9, 10, 11, 12, 13, 14, 15, 16, 17, 18, 29, 20, 21, 22, 23, 24, 25, 26, 27, 28, 29, 30, or 31 days apart, 1, 2, 3, 4, 5, 6, 7, 8, 9, 10, 11, or 12 months apart, or one or more years apart or any range therein. In some embodiments, the interval of time between administration of composition comprising rapamycin or an analog of rapamycin and the composition comprising the second active agent is 1 to 30 days.

In some aspects, the composition comprising rapamycin and/or an analog of rapamycin prevents or inhibits the growth of keloids and/or inhibits the development of adhesions or further development of existing adhesions. In some embodiments, the composition comprising rapamycin and/or an analog of rapamycin prevents the development of new keloids and/or adhesions, decreases the number or severity of keloids and/or adhesions, and/or induces a reduction in size or number of existing keloids and/or adhesions.

In some embodiments, the rapamycin or analog thereof are encapsulated or coated, or the composition comprising the rapamycin or analog thereof is encapsulated or coated. In some embodiments, the encapsulant or coating may be an enteric coating. In some embodiments, the mTOR inhibitor or an analog thereof is eRapa. “eRapa” is generically used to refer to encapsulated or coated forms of Rapamycin or other mTOR inhibitors or their respective analogs disclosed herein and equivalents thereof. In some embodiments, the encapsulant or coating used for and incorporated in eRapa preparation may be an enteric coating. In some embodiments, the mTOR inhibitor or analog thereof is nanoRapa. “nanoRapa” is generically used to refer to the rapamycins, rapamycin analogs, or related compositions within the eRapa preparation provided in the form of nanoparticles that include the rapamycin or other mTOR inhibitor. In some embodiments, the mTOR inhibitor or analog thereof is e-nanoRapa. “e-nanoRapa” is generically used to refer to eRapa variations formed from nanoRapa particles. After preparing the nanoRapa preparations, the nanoRapa preparation may then be coated with an enteric coating, to provide an eRapa preparation formed from nanoRapa particles.

In some embodiments, the eRapa, nanoRapa, or e-nanoRapa is encased in a coating comprising cellulose acetate succinate, hydroxy propyl methyl cellulose phthalate copolymer, or a polymethacrylate-based copolymer selected from the group consisting of methyl acrylate-methacrylic acid copolymer, and a methyl methacrylate-methacrylic acid copolymer. In some embodiments, the coating comprises Poly(methacylic acid-co-ethyl acrylate) in a 1:1 ratio, Poly(methacrylic acid-co-ethyl acrylate) in a 1:1 ratio, Poly(methacylic acid-co-methyl methacrylate) in a 1:1 ratio, Poly(methacylic acid-co-methyl methacrylate) in a 1:2 ratio, Poly(methyl acrylate-co-methyl methacrylate-co-methacrylic acid) in a 7:3:1 ratio, Poly(ethyl acrylate-co-methyl methacrylate-co-trimethylammonioethyl methacrylate chloride) in a 1:2:0.2 ratio, Poly(ethyl acrylate-co-methyl methacrylate-co-trimethylammonioethyl methacrylate chloride) in a 1:2:0.1 ratio, or Poly(butyl methacylate-co-(2-dimethylaminoethyl) methacrylate-co-methyl methacrylate) in a 1:2:1 ratio, a naturally-derived polymer, or a synthetic polymer, or any combination thereof. In some embodiments, the naturally-derived polymer is selected from the group consisting of alginates and their various derivatives, chitosans and their various derivatives, carrageenans and their various analogues, celluloses, gums, gelatins, pectins, and gellans. In some embodiments, the naturally-derived polymer is selected from the group consisting of polyethyleneglycols (PEGs) and polyethyleneoxides (PEOs), acrylic acid homo- and copolymers with acrylates and methacrylates, homopolymers of acrylates and methacrylates, polyvinyl alcohol PVOH), and polyvinyl pyrrolidone (PVP).

In some embodiments, the composition comprises eRapa or an analog thereof at a concentration of at or between 50 micrograms and 200 micrograms per kilogram for daily administration, or the equivalent for other frequencies of administration.

In some embodiments, the eRapa, nanoRapa, or e-nanoRapa is administered orally, enterically, colonically, anally, intravenously, or dermally with a patch. In some embodiments, the eRapa, nanoRapa, or e-nanoRapa is administered in two or more doses. In some embodiments, the interval of time between administration of doses comprising eRapa, nanoRapa, or e-nanoRapa is 0.5 to 30 days. In some embodiments, the interval of time between administration of doses comprising eRapa, nanoRapa, or e-nanoRapa is 0.5 to 1 day. In some embodiments, the interval of time between administration of doses comprising eRapa, nanoRapa, or e-nanoRapa is 1 to 3 days. In some embodiments, the interval of time between administration of doses comprising eRapa, nanoRapa, or e-nanoRapa is 1 to 5 days. In some embodiments, the interval of time between administration of doses comprising eRapa, nanoRapa, or e-nanoRapa is 1 to 7 days. In some embodiments, the interval of time between administration of doses comprising eRapa, nanoRapa, or e-nanoRapa is 1 to 15 days.

In some embodiments, the composition comprising eRapa, nanoRapa, or e-nanoRapa is comprised in a food or food additive.

Unless otherwise specified, the percent values expressed herein are weight by weight and are in relation to the total composition.

In embodiments of the disclosure, there is a method of a) preventing or reducing adhesion between two tissues and/or organs in an individual subjected to a procedure, and/or b) preventing or reducing one or more keloids in an individual subjected to a procedure; comprising the step of providing to the individual an effective amount of a composition comprising one or more inhibitors of an mTOR pathway no earlier than about 4 days following the procedure. In specific embodiments, the inhibitor of an mTOR pathway is rapamycin and/or a rapamycin analog. Examples of rapamycin analogs include temsirolimus, everolimus, deforolimus, CCI-779, curcumin, Green tea extract standardized to 70% EGCG, transresveratrol, fisetin, salicin extracted from white willow, or a combination thereof. In certain embodiments, the inhibitor of the mTOR pathway is an ATP-competitive mTOR kinase inhibitor, such as AZD8055, Torinl, PP242, PP30 or a combination thereof.

In embodiments of the disclosure, adhesions are between abdominal, pelvic, or thoracic organs and/or with the walls of the abdominal, pelvic, or thoracic cavities. Pelvic adhesions may involve a reproductive organ, the urinary bladder, the pelvic colon, and/or the rectum. Abdominal adhesions may involve the stomach, liver, gallbladder, spleen, pancreas, small intestine, kidney, large intestine, and/or adrenal gland.

Methods of the disclosure encompass any procedure that is an abdominal, thoracic or gynecological surgery, in certain embodiments.

In particular embodiments, the rapamycin or rapamycin analog is encased in a coating that comprises a cellulose acetate succinate or hydroxy propyl methyl cellulose phthalate co-polymer, or a polymethacrylate-based copolymer to include: methyl acrylate-methacrylic acid copolymer, or a methyl methacrylate-methacrylic acid copolymer. In specific embodiments, the coating comprises Poly(methacylic acid-co-ethyl acrylate) in a 1:1 ratio, Poly(methacrylic acid-co-ethyl acrylate) in a 1:1 ratio, Poly(methacylic acid-co-methyl methacrylate) in a 1:1 ratio, Poly(methacylic acid-co-methyl methacrylate) in a 1:2 ratio, Poly(methyl acrylate-co-methyl methacrylate-co-methacrylic acid) in a 7:3:1 ratio, Poly(ethyl acrylate-co-methyl methacrylate-co-trimethylammonioethyl methacrylate chloride) in a 1:2:0.2 ratio, Poly(ethyl acrylate-co-methyl methacrylate-co-trimethylammonioethyl methacrylate chloride) in a 1:2:0.1 ratio, or Poly(butyl methacylate-co-(2-dimethylaminoethyl) methacrylate-co-methyl methacrylate) in a 1:2:1 ratio, a naturally-derived polymer, or a synthetic polymer, or any combination thereof.

In embodiments wherein a naturally-derived polymer is employed, the naturally-derived polymer may include alginates and their various derivatives, chitosans and their various derivatives, carrageenans and their various analogues, celluloses, gums, gelatins, pectins, and/or gellans. In specific cases, the naturally-derived polymer is selected from the group consisting of polyethyleneglycols (PEGs) and polyethyleneoxides (PEOs), acrylic acid homo- and copolymers with acrylates and methacrylates, homopolymers of acrylates and methacrylates, polyvinyl alcohol PVOH), and polyvinyl pyrrolidone (PVP).

Embodiments of the disclosure include those wherein the mTOR inhibitor is provided to the individual topically and/or systemically. The inhibitor may be administered orally or enterically.

In particular embodiments, a composition comprises rapamycin or a rapamycin analog at a concentration of 0.001 mg to 30 mg total per dose. In some cases, the composition comprising rapamycin or an analog of rapamycin comprises 0.001% to 60% by weight of rapamycin or an analog of rapamycin. The composition may be administered in two or more doses and an example of an interval of time between administration of doses of the composition is 0.5 to 30 days, 0.5 to 1 day, 1 to 3 days, 1 to 7 days, 1-14 days. In specific embodiments, the composition is loaded into microparticles of a biodegradable polymer. The microparticles may be disposed within an encasing material formulated for enteric release. The rapamycin or rapamycin analog is predominantly released in the colon, in certain embodiments. The biodegradable polymer may comprise one or more of poly-ε-caprolactone, a polylactide, a polyglycolide, or combinations thereof. In specific ekmbodiments, an encasing material comprises a pH-dependent polymer that dissolves in a pH-dependent manner, and the pH-dependent polymer may comprised a methyl methacrylate-methacrylic acid copolymer (such as Eudragit S 100). In certain embodiments, the encasing material comprises a hydrophilic gelling polymer or copolymer, and the hydrophilic gelling polymer or copolymer may comprise one or more of methylcellulose, carboxymethylcellulose, hydroxypropylcellulose, hydroxypropylmethylcellulose, carbomers, polyvinyl alcohols, polyoxyethylene glycols, polyvinylpyrrolidones, poloxamers, or natural or synthetic rubbers.

In some embodiments, the encasing material comprises one or more of chitosan, pectin, or a combination thereof. The encasing material may comprise a starch capsule, such as one that comprises hydroxyethyl starch, hydroxypropyl starch, carboxymethyl starch, cationic starch, acetylated starch, phosphorylated starch, succinate derivatives, or grafted starches. In certain embodiments, the encasing material comprises a water-insoluble rupturable polymer layer (which may be semi-permeable), such as one that comprises cellulose acetate, cellulose acetate propionate, or ethyl cellulose.

In some embodiments, an effervescent material is disposed within the encasing material. In particular cases, the encasing material further comprises a swelling layer comprising croscarmellose sodium or hydroxyproplymethyl cellulose, and wherein the swelling layer is disposed within the rupturable polymer layer. In specific embodiments, a hydrophilic particulate material is embedded in the rupturable polymer layer, wherein the particulate material allows controlled entry of water past the rupturable polymer layer, wherein a swellable material is further disposed within the encasing material, and wherein the swellable material swells upon contact with water, causing the rupturable polymer layer to rupture. The encasing material may comprise a wax matrix, such as one that comprises behenic acid.

In particular embodiments, the encasing material comprises a first piece and a second piece, wherein the first piece contains an orifice, wherein the second piece is disposed initially to block the orifice and prevent entry of water, wherein the second piece comprises a swellable material, and wherein contacting the second piece with water causes it to swell and become displaced from the orifice. In specific examples, the composition is administered regularly for more than a week, more than a month, more than six months, more than one year, more than two years, more than three years, more than four years, or more than five years. In particular embodiments, the individual is provided an effective amount of the composition no earlier than about 3 days following the procedure. In specific embodiments, the individual is provided an effective amount of the composition no earlier than about 48 hours following the procedure.

In some embodiments, there is disclosed a pharmaceutical composition for treating or preventing adhesions or keloids comprising microparticles of a biodegradable polymer loaded with rapamycin or a rapamycin analog, wherein the microparticles are disposed within an encasing material formulated for enteric release.

In certain embodiments, there is a method of a) preventing or reducing adhesion between two tissues and/or organs in an individual subjected to a procedure, and/or b) preventing or reducing one or more keloids in an individual subjected to a procedure; comprising the step of providing to the individual an effective amount of a composition comprising one or more inhibitors of an mTOR pathway no earlier than about 4 days following the procedure. In particular embodiments, the inhibitor of an mTOR pathway is rapamycin and/or a rapamycin analog. In specific embodiments, the rapamycin analog is selected from the group consisting of temsirolimus, everolimus, deforolimus, CCI-779, curcumin, Green tea extract standardised to 70% EGCG, transresveratrol, fisetin, salicin extracted from white willow, and a combination thereof. The inhibitor of the mTOR pathway may be an ATP-competitive mTOR kinase inhibitor, in certain embodiments, and the the ATP-competitive mTOR kinase inhibitor may be selected from the group consisting of AZD8055, Torinl, PP242, PP30 and a combination thereof.

In particular embodiments of the disclosure, adhesions are between abdominal, pelvic, or thoracic organs and/or with the walls of the abdominal, pelvic, or thoracic cavities. In specific embodiments, pelvic adhesions involve a reproductive organ, the urinary bladder, the pelvic colon, and/or the rectum and/or abdominal adhesions involve the stomach, liver, gallbladder, spleen, pancreas, small intestine, kidney, large intestine, and/or adrenal gland. In specific embodiments, the procedure is an abdominal, thoracic or gynecological surgery.

In particular embodiments, a rapamycin or rapamycin analog is encased in a coating that comprises a cellulose acetate succinate or hydroxy propyl methyl cellulose phthalate co-polymer, or a polymethacrylate-based copolymer to include: methyl acrylate-methacrylic acid copolymer, or a methyl methacrylate-methacrylic acid copolymer. In specific embodiments, the coating comprises Poly(methacylic acid-co-ethyl acrylate) in a 1:1 ratio, Poly(methacrylic acid-co-ethyl acrylate) in a 1:1 ratio, Poly(methacylic acid-co-methyl methacrylate) in a 1:1 ratio, Poly(methacylic acid-co-methyl methacrylate) in a 1:2 ratio, Poly(methyl acrylate-co-methyl methacrylate-co-methacrylic acid) in a 7:3:1 ratio, Poly(ethyl acrylate-co-methyl methacrylate-co-trimethylammonioethyl methacrylate chloride) in a 1:2:0.2 ratio, Poly(ethyl acrylate-co-methyl methacrylate-co-trimethylammonioethyl methacrylate chloride) in a 1:2:0.1 ratio, or Poly(butyl methacylate-co-(2-dimethylaminoethyl) methacrylate-co-methyl methacrylate) in a 1:2:1 ratio, a naturally-derived polymer, or a synthetic polymer, or any combination thereof. A naturally-derived polymer may be selected from the group consisting of alginates and their various derivatives, chitosans and their various derivatives, carrageenans and their various analogues, celluloses, gums, gelatins, pectins, and gellans. A naturally-derived polymer may be selected from the group consisting of polyethyleneglycols (PEGs) and polyethyleneoxides (PEOs), acrylic acid homo- and copolymers with acrylates and methacrylates, homopolymers of acrylates and methacrylates, polyvinyl alcohol PVOH), and polyvinyl pyrrolidone (PVP).

In particular embodiments, an inhibitor of an mTOR pathway is provided to an individual topically, systemically, orally, or enterically. In a specific embodiment, the composition comprises rapamycin or a rapamycin analog at a concentration of 0.001 mg to 30 mg total per dose. In certain embodiments, a composition comprising rapamycin or an analog of rapamycin comprises 0.001% to 60% by weight of rapamycin or an analog of rapamycin. Any composition may be administered in two or more doses. In specific embodiments, the interval of time between administration of doses of the composition is 0.5 to 30 days, 0.5 to 1 day, 1 to 3 days, 1 to 7 days, 1-14 days, and so forth.

A composition encompassed by the disclosure may be loaded into microparticles of a biodegradable polymer. In specific embodiments, the microparticles are disposed within an encasing material formulated for enteric release, such as predominantly released in the colon, as an example. In specific embodiments, the biodegradable polymer comprises one or more of poly-ε-caprolactone, a polylactide, a polyglycolide, or combinations thereof. In particular embodiments, the encasing material comprises a pH-dependent polymer that dissolves in a pH-dependent manner, such as a methyl methacrylate-methacrylic acid copolymer, for example, including Eudragit S 100. In certain embodiments, the encasing material comprises a hydrophilic gelling polymer or copolymer, such as one or more of methylcellulose, carboxymethylcellulose, hydroxypropylcellulose, hydroxypropylmethylcellulose, carbomers, polyvinyl alcohols, polyoxyethylene glycols, polyvinylpyrrolidones, poloxamers, or natural or synthetic rubbers. In specific embodiments, the encasing material comprises one or more of chitosan, pectin, or a combination thereof. An encasing material encompassed by the disclosure may comprise a starch capsule, such as one that comprises hydroxyethyl starch, hydroxypropyl starch, carboxymethyl starch, cationic starch, acetylated starch, phosphorylated starch, succinate derivatives, or grafted starches. In specific embodiments, the encasing material comprises a water-insoluble rupturable polymer layer, such as one that comprises cellulose acetate, cellulose acetate propionate, or ethyl cellulose. The rupturable polymer layer may be semi-permeable, in specific embodiments. In specific embodiments, an effervescent material is disposed within the encasing material. The encasing material may further comprise a swelling layer comprising croscarmellose sodium or hydroxyproplymethyl cellulose, and wherein the swelling layer is disposed within the rupturable polymer layer. In specific embodiments, a hydrophilic particulate material is embedded in the rupturable polymer layer, wherein the particulate material allows controlled entry of water past the rupturable polymer layer, wherein a swellable material is further disposed within the encasing material, and wherein the swellable material swells upon contact with water, causing the rupturable polymer layer to rupture. In specific embodiments, an encasing material comprises a wax matrix, such as one that comprises behenic acid.

In some embodiments, an encasing material comprises a first piece and a second piece, wherein the first piece contains an orifice, wherein the second piece is disposed initially to block the orifice and prevent entry of water, wherein the second piece comprises a swellable material, and wherein contacting the second piece with water causes it to swell and become displaced from the orifice.

In some embodiments, the composition is administered regularly for more than a week, more than a month, more than six months, more than one year, more than two years, more than three years, more than four years, or more than five years. In specific embodiments, an individual is provided an effective amount of the composition no earlier than about 3 days following the procedure. In certain embodiments, an individual is provided an effective amount of the composition no earlier than about 48 hours following the procedure.

It is contemplated that any embodiment discussed in this specification can be implemented with respect to any method or composition of the invention, and vice versa. Furthermore, compositions of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWING

FIG. 1 demonstrates a calculated surface area in cm² of a keloid to which RAPA ointment had been applied twice a day. Linear regression lines are shown for months 4-7.

### DETAILED DESCRIPTION

The term “about” or “approximately” are defined as being close to as understood by one of ordinary skill in the art, and in one non-limiting embodiment the terms are defined to be within 10%, preferably within 5%, more preferably within 1%, and most preferably within 0.5%.

The terms “inhibiting,” “reducing,” “treating,” or any variation of these terms, includes any measurable decrease or complete inhibition to achieve a desired result. Similarly, the term “effective” means adequate to accomplish a desired, expected, or intended result.

The terms “prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

The use of the word “a” or “an” when used in conjunction with the term “comprising” may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. in relation to the total composition.

The compositions and methods for their use can “comprise,” “consist essentially of,” or “consist of” any of the ingredients or steps disclosed throughout the specification. With respect to the transitional phrase “consisting essentially of,” in one non-limiting aspect, a basic and novel characteristic of the compositions and methods is the ability of eRapa, e-nanoRapa, or other rapamycin preparations to prevent or inhibit the growth of endocrine-related adenomas, neoplasia, or dysplasia in a patient who has been identified as being at risk for developing an endocrine tumor or endocrine cancer.

The term “procedure” or “medical procedure” as used herein refers to any medical event in which the body of an individual is subjected to cutting through of one or more tissues and/or organs. In specific embodiments, the tissue and/or organ is in the abdominal, pelvic, or thoracic region of an individual, including in the walls of the abdominal, pelvic, or thoracic cavities, for example. Tissues and/or organs include those of a reproductive organ, the urinary bladder, the pelvic colon, the rectum, the stomach, the liver, the gallbladder, the spleen, the pancreas, the small intestine, the kidney, the large intestine, and/or the adrenal gland.

“Treatment” and “treating” refer to administration or application of a therapeutic agent to a subject or performance of a procedure or modality on a subject for the purpose of obtaining a therapeutic benefit for a disease or health-related condition. For example, the rapamycin compositions of the present invention may be administered to a subject for the purpose of treating or preventing intestinal adenomas or polyps and cancer in a patient who has been identified as being at risk for developing intestinal polyps or intestinal cancer.

The terms “therapeutic benefit,” “therapeutically effective,” or “effective amount” refer to the promotion or enhancement of the well-being of a subject. This includes, but is not limited to, a reduction in the frequency or severity of the signs or symptoms of a disease.

“Prevention” and “preventing” are used according to their ordinary and plain meaning. In the context of a particular disease or health-related condition, those terms refer to administration or application of an agent, drug, or remedy to a subject or performance of a procedure or modality on a subject for the purpose of preventing or delaying the onset of a disease or health-related condition.

I. Embodiments of Treatment and/or Prevention Methods

Adhesions are fibrous bands of internal scar tissue that form between tissues and organs, typically following abdominal or gynecological surgeries. Adhesions develop after nearly every abdominal surgery, they begin forming within hours and within a few weeks cause internal organs to attach to the surgical site or to other organs in the abdominal cavity, frequently resulting in abdominal pain or intestinal obstruction. Common adhesion-related complications include small bowel obstruction, female infertility, and chronic pelvic pain. In patients who have abdominal surgery, 93% will develop adhesions that may require a second operation to break the adhesions. Nearly 350,000 procedures are performed yearly to lyse peritoneal adhesions. Even after adhesion lysis, recurrent obstruction and re-operation is common, further adding to the physical, emotional, and financial costs. The prevention of intra-abdominal and pelvic adhesions could save billions of dollars in health care costs and improve the lives of hundreds of thousands of patients. Clearly, abdominal adhesions are a clinically and financially significant problem.

Tulandi, et al. (2011) sought to evaluate postsurgical adhesions in women of different races with or without keloids. They prospectively evaluated postsurgical adhesions after a cesarean delivery in 429 women with or without keloids. The outcome measures were the prevalence and extent of adhesions in women of different races with or without keloids. They found that the prevalence and degree of postsurgical adhesions in women of different races were comparable; however, women with keloids had increased adhesions between the uterus and the bladder and between the uterus and the abdominal wall. Their findings indicate that keloid prone patients are also at increased risk for postoperative adhesion development.

Keloids are similar to intra-abdominal adhesions in that they do not regress spontaneously and they tend to recur after excision. Furthermore, keloids are histologically similar to intra-abdominal adhesions. In both lesions there is excess production and deposition of extracellular matrix, especially collagen and fibronectin. Compared to normal tissue, both express a number of genes that regulate cell growth and apoptosis, inflammation, angiogenesis, and tissue turnover. Epidemiological work found that individuals with keloids had more intra-abdominal adhesions between the uterus and bladder and between the uterus and anterior abdominal wall than those without keloids (Tulandi et al., 2011). This suggests that individuals with keloids are prone to develop intra-abdominal adhesions.

Dietrich et al. (2012) attempted to prevent post-surgical adhesions in a rat model by daily IP injection of RAPA immediately following surgery. They found that postoperative RAPA treatment led to enhanced adhesion development and a higher rate of wound infections. The inventor believes that the timing and method of application of RAPA administration confounded these results. Daily IP injections would cause increased intraabdominal trauma and promote inflammatory changes in the serosa. It is known that inflammatory processes contribute to adhesion formation. Indeed, Hellebrekers et al. (Hellebrekers and Kooistra, 2011) found that peritoneal inflammatory status is a crucial factor in determining the balance between fibrin formation and dissolution, thus determining whether or not adhesions develop. In addition, the assessment of adhesions used by Dietrich et al. (2012) was based primarily on gross examination and occurred 3 months post-surgery—8 weeks after stopping RAPA treatment—which may not be appropriate to systemically assess the effects of RAPA on adhesion processes, especially at early stages.

Surgeons have been concerned about using RAPA in the post-operative period because RAPA retards wound healing. Willems et al. (2011) addressed this concern and found that administration of RAPA immediately following surgery led to serious loss of wound strength, both in the intestine and in the abdominal fascia. Importantly, Willems et al. also found that delaying administration of RAPA for 2-4 days post-surgery ameliorated this loss.

Methods of the disclosure concern treatment and/or prevention of adhesions and/or keloids and/or fibrosis in an individual subjected to a procedure in which at least one tissue and/or organ is exposed to cutting, such as cut surgically, for example.

In particular embodiments, following a sufficient time after a procedure the individual is provided an effective amount of the rapamycin and/or rapamycin analog that reduces the size of at least one adhesion in the individual and/or that prevents the formation of at least one adhesion in the individual and/or that prevents the enlargement of at least one adhesion in the individual.

In embodiments of the disclosure, the formation of one or more keloids is prevented or one or more existing keloids are reduced in size in an individual subjected to a procedure. The keloid may or may not be a result of the procedure. In cases where a keloid occurs following a procedure, the rapamycin and/or rapamycin analog may be provided following a sufficient time after the procedure.

In methods of the disclosure, any deleterious impact on wound healing following a procedure is avoided by waiting a specific period of time before the rapamycin and/or rapamycin analog is provided to the individual. In particular embodiments, a delay of at least 2 days, 3, days, 4 days, 5 days, or 6 days are more occurs before the individual is provided the effective amount of rapamycin and/or rapamycin analog. In particular embodiments, a delay of at least 24-150 hours occurs before the individual is provided an effective amount of rapamycin and/or rapamycin analog. As a result, the wound may heal to a sufficient extent such that the rapamycin or rapamycin analog does not deleteriously impact the healing of the wound.

Any mTOR composition of the disclosure may be provided to the individual upon a sufficient time following the procedure either systemically or locally.

In alternative embodiments, the tissue and/or organ is subjected to cutting into or through not as a result of a planned procedure but as a result of trauma or accident, for example. In such case, the individual is provided a mTOR inhibitor, such as rapamycin and/or a rapamycin analog, no earlier than 2, 3, 4, 5, or 6 or more days after the trauma or accident. In some embodiments, methods are utilized to reduce scar formation, adhesion formation, keloid formation, etc., due to any inflammatory process including those induced by infectious processes that cause tissue inflammation or trauma that causes tissue inflammation.

Post-operative intra-abdominal adhesions are a common complication of wound healing following surgical and gynecological procedures. Such post-operative complications result in significant morbidity and even mortality. For example, more than 50% of small bowel obstructions are attributable to prior surgical procedures. Adhesion formation also contributes to infertility and chronic pelvic pain. Re-operation with surgical removal of adhesions accounts for more than 50% of all repeated laparotomies. Recurrence of adhesions after repeated laparotomies, i.e., treatment failure, is not uncommon. Embodiments of the disclosure provide immense clinical benefit by disclosing preventative and/or non-operative treatments for post-operative intra-abdominal adhesions. While use of rapamycin or other mTOR antagonists appears to be mechanistically promising as such a treatment, mTOR antagonists are known to retard wound healing and thereby reduce the strength of wound scar tissue if applied immediately after wounding. Delaying mTOR antagonism for several days (2, 3, or 4 or more) post-surgery will obviate this problem, resulting in reduced adhesion formation/fibrosis and yet permit adequate wound healing.

II. mTOR Inhibitors, Rapamycin and Rapamycin Analogs

Any inhibitor of mTOR is contemplated for inclusion in the present compositions and methods. In particular embodiments, the inhibitor of mTOR is rapamycin or an analog of rapamycin. Rapamycin (also known as sirolimus and marketed under the trade name Rapamune®) is a known macrolide. The molecular formula of rapamycin is C₅₁H₇₉NO₁₃. The chemical name is (3S,6R,7E,9R,10R,12R,14S,15E,17E,19E,21S,23S,26R,27R,34aS)-9,10,12,13,14,-21,22,23,24,25,26,27,32,33,34,34a-hexadecahydro-9,27-dihydroxy-3-[(1R)-2-[-(1S,3R,4R)-4-hydroxy-3-methoxycyclohexyl]-1-methylethyl]-10,21-dimethoxy-6,8,12,14,20,26-hexamethyl-23,27-epoxy-3H-pyrido[2,1-c][1,4]oxaazacyclohent-ria-contine-1,5,11,28,29(4H,6H,31H)-pentone.

Rapamycin binds to a member of the FK binding protein (FKBP) family, FKBP 12. The rapamycin/FKBP 12 complex binds to the protein kinase mTOR to block the activity of signal transduction pathways. Because the mTOR signaling network includes multiple tumor suppressor genes, including PTEN, LKB1, TSC1, and TSC2, and multiple proto-oncogenes including PI3K, Akt, and eEF4E, mTOR signaling plays a central role in cell survival and proliferation. Binding of the rapamycin/FKBP complex to mTOR causes arrest of the cell cycle in the G1 phase (Janus et al., 2005).

mTOR inhibitors also include rapamycin analogs. Many rapamycin analogs are known in the art. Non-limiting examples of analogs of rapamycin include, but are not limited to, everolimus, tacrolimus, CCI-779, ABT-578, AP-23675, AP-23573, AP-23841, 7-epi-rapamycin, 7-thiomethyl-rapamycin, 7-epi-trimethoxyphenyl-rapamycin, 7-epi-thiomethyl-rapamycin, 7-demethoxy-rapamycin, 32-demethoxy-rapamycin, 2-desmethyl-rapamycin, prerapamycin, temsirolimus, and 42-O-(2-hydroxy)ethyl rapamycin.

Other analogs of rapamycin include at least the following: rapamycin oximes (U.S. Pat. No. 5,446,048); rapamycin aminoesters (U.S. Pat. No. 5,130,307); rapamycin dialdehydes (U.S. Pat. No. 6,680,330); rapamycin 29-enols (U.S. Pat. No. 6,677,357); O-alkylated rapamycin derivatives (U.S. Pat. No. 6,440,990); water soluble rapamycin esters (U.S. Pat. No. 5,955,457); alkylated rapamycin derivatives (U.S. Pat. No. 5,922,730); rapamycin amidino carbamates (U.S. Pat. No. 5,637,590); biotin esters of rapamycin (U.S. Pat. No. 5,504,091); carbamates of rapamycin (U.S. Pat. No. 5,567,709); rapamycin hydroxyesters (U.S. Pat. No. 5,362,718); rapamycin 42-sulfonates and 42-(N-carbalkoxy)sulfamates (U.S. Pat. No. 5,346,893); rapamycin oxepane isomers (U.S. Pat. No. 5,344,833); imidazolidyl rapamycin derivatives (U.S. Pat. No. 5,310,903); rapamycin alkoxyesters (U.S. Pat. No. 5,233,036); rapamycin pyrazoles (U.S. Pat. No. 5,164,399); acyl derivatives of rapamycin (U.S. Pat. No. 4,316,885); reduction products of rapamycin (U.S. Pat. Nos. 5,102,876 and 5,138,051); rapamycin amide esters (U.S. Pat. No. 5,118,677); rapamycin fluorinated esters (U.S. Pat. No. 5,100,883); rapamycin acetals (U.S. Pat. No. 5,151,413); oxorapamycins (U.S. Pat. No. 6,399,625); and rapamycin silyl ethers (U.S. Pat. No. 5,120,842), each of which is specifically incorporated by reference.

Other analogs of rapamycin include those described in U.S. Pat. Nos. 7,560,457; 7,538,119; 7,476,678; 7,470,682; 7,455,853; 7,446,111; 7,445,916; 7,282,505; 7,279,562; 7,273,874; 7,268,144; 7,241,771; 7,220,755; 7,160,867; 6,329,386; RE37,421; 6,200,985; 6,015,809; 6,004,973; 5,985,890; 5,955,457; 5,922,730; 5,912,253; 5,780,462; 5,665,772; 5,637,590; 5,567,709; 5,563,145; 5,559,122; 5,559,120; 5,559,119; 5,559,112; 5,550,133; 5,541,192; 5,541,191; 5,532,355; 5,530,121; 5,530,007; 5,525,610; 5,521,194; 5,519,031; 5,516,780; 5,508,399; 5,508,290; 5,508,286; 5,508,285; 5,504,291; 5,504,204; 5,491,231; 5,489,680; 5,489,595; 5,488,054; 5,486,524; 5,486,523; 5,486,522; 5,484,791; 5,484,790; 5,480,989; 5,480,988; 5,463,048; 5,446,048; 5,434,260; 5,411,967; 5,391,730; 5,389,639; 5,385,910; 5,385,909; 5,385,908; 5,378,836; 5,378,696; 5,373,014; 5,362,718; 5,358,944; 5,346,893; 5,344,833; 5,302,584; 5,262,424; 5,262,423; 5,260,300; 5,260,299; 5,233,036; 5,221,740; 5,221,670; 5,202,332; 5,194,447; 5,177,203; 5,169,851; 5,164,399; 5,162,333; 5,151,413; 5,138,051; 5,130,307; 5,120,842; 5,120,727; 5,120,726; 5,120,725; 5,118,678; 5,118,677; 5,100,883; 5,023,264; 5,023,263; 5,023,262; all of which are incorporated herein by reference. Additional rapamycin analogs and derivatives can be found in the following U.S. Patent Application Pub. Nos., all of which are herein specifically incorporated by reference: 20080249123, 20080188511; 20080182867; 20080091008; 20080085880; 20080069797; 20070280992; 20070225313; 20070203172; 20070203171; 20070203170; 20070203169; 20070203168; 20070142423; 20060264453; and 20040010002.

Rapamycin or a rapamycin analogs can be obtained from any source known to those of ordinary skill in the art. The source may be a commercial source, or natural source. Rapamycin or a rapamycin analog may be chemically synthesized using any technique known to those of ordinary skill in the art. Non-limiting examples of information concerning rapamycin synthesis can be found in Schwecke et al., 1995; Gregory et al., 2004; Gregory et al., 2006; Graziani, 2009.

Additional therapeutic agents, or any number of additional adjunct ingredients, may be included with the rapamycin and/or rapamycin analog. For example, the core may further include at least one of an absorption enhancer, a binder, a hardness enhancing agent, a filler, a disintegrant, stabilizer, lubricant, chelating agent, an excipient, an antioxidant, and so forth. Examples of binders include povidone (PVP: polyvinyl pyrrolidone), low molecular weight HPC (hydroxypropyl cellulose), low molecular weight HPMC (hydroxypropyl methylcellulose), low molecular weight carboxy methyl cellulose, ethylcellulose, gelatin polyethylene oxide, acacia, dextrin, magnesium aluminum silicate, starch, and polymethacrylates. Examples of stabilizers include at least one of butyl hydroxyanisole, ascorbic acid and citric acid. Examples of disintegrants are selected from the group consisting of croscarmellose sodium, crospovidone (cross-linked polyvinyl pyrolidone) sodium carboxymethyl starch (sodium starch glycolate), cross-linked sodium carboxymethyl cellulose (Croscarmellose), pregelatinized starch (starch 1500), microcrystalline starch, water insoluble starch, calcium carboxymethyl cellulose, magnesium aluminum silicate and a combination thereof. A filler may be included, such as monohydrate, microcrystalline cellulose, starch, lactitol, lactose, a suitable inorganic calcium salt, sucrose, or a combination thereof. Antioxidants may be selected from the group consisting of 4,4 (2,3 dimethyl tetramethylene dipyrochatechol), Tocopherol-rich extract (natural vitamin E), .alpha.-tocopherol (synthetic Vitamin E), β-tocopherol, γ-tocopherol, Δ-tocopherol, Butylhydroxinon, Butyl hydroxyanisole (BHA), Butyl hydroxytoluene (BHT), Propyl Gallate, Octyl gallate, Dodecyl Gallate, Tertiary butylhydroquinone (TBHQ), Fumaric acid, Malic acid, Ascorbic acid (Vitamin C), Sodium ascorbate, Calcium ascorbate, Potassium ascorbate, Ascorbyl palmitate, Ascorbyl stearate, Citric acid, Sodium lactate, Potassium lactate, Calcium lactate, Magnesium lactate, Anoxomer, Erythorbic acid, Sodium erythorbate, Erythorbin acid, Sodium erythorbin, Ethoxyquin, Glycine, Gum guaiac, Sodium citrates (monosodium citrate, disodium citrate, trisodium citrate), Potassium citrates (monopotassium citrate, tripotassium citrate), Lecithin, Polyphosphate, Tartaric acid, Sodium tartrates (monosodium tartrate, disodium tartrate), Potassium tartrates (monopotassium tartrate, dipotassium tartrate), Sodium potassium tartrate, Phosphoric acid, Sodium phosphates (monosodium phosphate, disodium phosphate, trisodium phosphate), Potassium phosphates (monopotassium phosphate, dipotassium phosphate, tripotassium phosphate), Calcium disodium ethylene diamine tetra-acetate (Calcium disodium EDTA), Lactic acid, Trihydroxy butyrophenone and Thiodipropionic acid. Chelating agents may be included such as antioxidants, dipotassium edentate, disodium edentate, edetate calcium disodium, edetic acid, fumaric acid, malic acid, maltol, sodium edentate, trisodium edetate. Lubricants include stearate salts; stearic acid, corola oil, glyceryl palmitostearate, hydrogenated vegetable oil, magnesium oxide, mineral oil, poloxamer, polyethylene glycole, polyvinyl alcohol, magnesium stearate, sodium benzoate, talc, sodium stearyl fumarate, compritol (glycerol behenate), and sodium lauryl sulfate (SLS) or a combination thereof. A composition comprising rapamycin and/or rapamycin analog may contain a hydrophilic, swellable, hydrogel-forming material, such as one covered by a coating that includes a water insoluble polymer and hydrophilic water permeable agent, through which water enters the core. The swellable hydrogel-forming material in the core then swells and bursts the coating, after which the core more preferably disintegrates slowly or otherwise releases the rapamycin and/or rapamycin analog. Another embodiment relates to a release-controlling region comprising rapamycin and/or rapamycin analog with an slow-erodible dry coating.

### III. Encased, Encapsulated, or Coated Rapamycin/Rapamycin Analog Compositions

In some embodiments, the rapamycin and/or rapamycin analog composition (or any mTOR inhibitor) is formulated for use such that the composition is not deleteriously affected upon delivery through a particular delivery route, such as being exposed to at least part of the alimentary canal. Many pharmaceutical dosage forms irritate the stomach, for example, because of their chemical properties or are degraded by stomach acid through the action of enzymes, thus becoming less effective. Therefore, in specific embodiments, the rapamycin and/or rapamycin analog composition may be coated.

The coating may be an enteric coating, a coating that prevents release and absorption of active ingredients until they reach the intestine. “Enteric” refers to the small intestine, and therefore enteric coatings facilitate delivery of agents to the small intestine. Some enteric coatings facilitate delivery of agents to the colon. In some embodiments, the enteric coating is a EUDRAGIT® coating. Eudragit coatings include Eudragit L100-44 (for delivery to the duodenum), Eudragit L 30 D-55 (for delivery to the duodenum), Eudragit L 100 (for delivery to the jejunum), Eudragit S100 (for delivery to the ileum), and Eudragit FS 30D (for colon delivery). Poly(methyl acrylate-co-methyl methacrylate-co-methacrylic acid) 7:3:1; Eudragit RL (for sustained release), Poly(ethyl acrylate-co-methyl methacrylate-co-trimethylammonioethyl methacrylate chloride) 1:2:0.2; Eudragit RS (for sustained release), Poly(ethyl acrylate-co-methyl methacrylate-co-trimethylammonioethyl methacrylate chloride) 1:2:0.1; and Eudragit E (for taste masking), Poly(butyl methacylate-co-(2-dimethylaminoethyl) methacrylate-co-methyl methacrylate) 1:2:1. Other coatings include Eudragit RS, Eudragit RL, ethylcellulose, and polyvinyl acetate. Benefits include pH-dependent drug release, protection of active agents sensitive to gastric fluid, protection of gastric mucosa from active agents, increase in drug effectiveness, good storage stability, and GI and colon targeting, which minimizes risks associated with negative systemic effects. A variety of other encasing materials and systems for delivering rapamycin-loaded biodegradable microspheres to the colon can be used alone or in combination with a pH-dependent coating like Eudragit S100.

Hydrophilic gelling polymers or copolymers can be included in a material encasing one or more microspheres to provide a time-dependent release of drug-loaded microspheres. Non-limiting examples of hydrophilic gelling copolymers include methylcellulose, carboxymethylcellulose, hydroxypropylcellulose, hydroxypropylmethylcellulose, carbomers, polyvinyl alcohols, polyoxyethylene glycols, polyvinylpyrrolidones, poloxamers, or natural or synthetic rubbers. An intermediate layer of these polymers can be included to delay release of active ingredient for a desired amount of time, as described in Poli et al. (EP0572942). Another example of a time-dependent encasing material is a wax matrix including, for example, behenic acid, as described in Otsuka & Matsuda, 1994. Some examples of enteric coating components include cellulose acetate pthalate, methyl acrylate-methacrylic acid copolymers, cellulose acetate succinate, hydroxy propyl methyl cellulose phthalate, hydroxy propyl methyl cellulose acetate succinate, polyvinyl acetate phthalate, methyl methacrylate-methacrylic acid copolymers, sodium alginate, and stearic acid. The coating may include suitable hydrophilic gelling polymers including but not limited to cellulosic polymers, such as methylcellulose, carboxymethylcellulose, hydroxypropylcellulose, hydroxypropylmethylcellulose, hydroxyethylcellulose, and the like; vinyl polymers, such as polyvinylpyrrolidone, polyvinyl alcohol, and the like; acrylic polymers and copolymers, such as acrylic acid polymer, methacrylic acid copolymers, ethyl acrylate-methyl methacrylate copolymers, natural and synthetic gums, such as guar gum, arabic gum, xanthan gum, gelatin, collagen, proteins, polysaccharides, such as pectin, pectic acid, alginic acid, sodium alginate, polyaminoacids, polyalcohols, polyglycols; and the like; and mixtures thereof. Any other coating agent known to those of ordinary skill in the art is contemplated for inclusion in the coatings of the microcapsules set forth herein.

The coating may optionally comprises a plastisizer, such as dibutyl sebacate, polyethylene glycol and polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol or a combination thereof. The coating may optionally include a gum. Non-limiting examples of gums include homopolysaccharides such as locust bean gum, galactans, mannans, vegetable gums such as alginates, gum karaya, pectin, agar, tragacanth, accacia, carrageenan, tragacanth, chitosan, agar, alginic acid, other polysaccharide gums (e.g., hydrocolloids), acacia catechu, salai guggal, indian bodellum, copaiba gum, asafetida, cambi gum, Enterolobium cyclocarpum, mastic gum, benzoin gum, sandarac, gambier gum, butea frondosa (Flame of Forest Gum), myrrh, konjak mannan, guar gum, welan gum, gellan gum, tara gum, locust bean gum, carageenan gum, glucomannan, galactan gum, sodium alginate, tragacanth, chitosan, xanthan gum, deacetylated xanthan gum, pectin, sodium polypectate, gluten, karaya gum, tamarind gum, ghatti gum, Accaroid/Yacca/Red gum, dammar gum, juniper gum, ester gum, ipil-ipil seed gum, gum talha (acacia seyal), and cultured plant cell gums including those of the plants of the genera: acacia, actinidia, aptenia, carbobrotus, chickorium, cucumis, glycine, hibiscus, hordeum, letuca, lycopersicon, malus, medicago, mesembryanthemum, oryza, panicum, phalaris, phleum, poliathus, polycarbophil, sida, solanum, trifolium, trigonella, Afzelia africana seed gum, Treculia africana gum, detarium gum, cassia gum, carob gum, Prosopis africana gum, Colocassia esulenta gum, Hakea gibbosa gum, khaya gum, scleroglucan, zea, mixtures of any of the foregoing, and the like.

Polysaccharides that are resistant to digestive enzymes but are enzymatically broken down by bacteria in the colon can be included in an encasing material. Non-limiting examples include chitosan and pectin as described in Coulter (EP2380564), and azopolymers, disulfide polymers, amylose, calcium pectinate, and chondroitin sulfate as described in Watts (EP0810857).

A starch capsule coated with an enteric coating such as Eudragit S 100 or Eudragit L 100 may be used, as described in Watts (EP0810857). A variety of starches, including modified starches and starch derivatives may be used. Non-limiting examples include hydroxyethyl starch, hydroxypropyl starch, carboxymethyl starch, cationic starch, acetylated starch, phosphorylated starch, succinate derivatives, or grafted starches.

A layer of insoluble, or relatively insoluble rupturable polymer can be used as part of a strategy to provide for abrupt release of drug-loaded microspheres in the colon. The rupturable polymer can comprise one or more of a variety of suitable polymers known by those of skill in the art, including but not limited to cellulose acetate, cellulose acetate propionate, or ethyl cellulose. A variety of strategies for causing rupture of the polymer in the colon can be employed. As a non-limiting example, the rupturable polymer can be designed to rupture upon encountering increased pressure due to intestinal peristalsis, as described in Muraoka et al., 1998. As another example, the rupturable polymer can be semi-permeable, and an effervescent solid can be included in a core containing the drug-loaded microparticles, as described in Krogel & Bodmeier, 1999. As another example, a layer of swellable material, including but not limited to croscarmellose sodium or hydroxyproplymethyl cellulose, can be disposed within the rupturable polymer layer, as described in Bussemer, et al., 2001. Controlled entry of water past the rupturable polymer layer can be provided by embedded hydrophilic particulate material, as described in Lerner et al. (WIPO Pub. No. WO 1999/018938).

In specific embodiments, applying an enteric coating involves use of a spinning disk atomizer, other methods may include pan coating, air-suspension coating, centrifugal extrusion, vibrational nozzle, spray-drying, interfacial polymerization, in situ polymerization, matrix polymerization.

A two-piece encasing system, as described in McNeill et al. WIPO Pub. No. WO 1990/009168 can be used to provide for release of drug-loaded microspheres in the colon. One of the pieces is a relatively water insoluble capsule with an open orifice, which is covered by a second piece that swells as it takes up water. The swelling causes displacement from the orifice and release of the capsule contents.

### IV. Encapsulated Rapamycin Compositions

In some aspects, the compositions comprising an inhibitor of mTOR are encapsulated or coated to provide eRapa preparations. In some embodiments, the encapsulant or coating may be an enteric coating. In some embodiments, the compositions comprising an inhibitor of mTOR are provided in the form of nanoRapa nanoparticles, and such nanoRapa nanoparticles are encapsulated or coated to provide e-nanoRapa preparations, which are relatively stable and beneficial for oral administration.

In some aspects, the compositions comprising an inhibitor of mTOR are formed into nanoparticles and subsequently encapsulated or coated. In some embodiments, the encapsulant or coating may be an enteric coating. In some embodiments, the encapsulated rapamycin nanoparticles provide rapamycin nanoparticles within a protective polymer matrix for oral administration of rapamycin. The result is not only more durable and stable, but is also more bioavailable and efficacious for treatment and prevention of genetically-predisposed disorders and age-related disorders, especially in the fields of oncology and neurology in humans and other animals.

The encapsulated rapamycin nanoparticles provide an embodiment of the present invention in the form of an improved form of encapsulated rapamycin that is more durable, stable and bioavailable. In some embodiments, the encapsulated rapamycin provides the rapamycin nanoparticles within a controlled release matrix, forming the encapsulated rapamycin nanoparticle in a single drug delivery structure for oral administration of rapamycin. This encapsulated rapamycin nanoparticle may also be referred to as an enteric-coated rapamycin nanoparticle. In addition, many of the embodiments also include a stabilizing compound (for these purposes, a “stabilizer”) within the controlled release matrix either to improve compatibility of the rapamycin with the controlled release matrix, to stabilize the crystalline morphology of the rapamycin, or to help further prevent degradation of the rapamycin, particularly when the encapsulated rapamycin nanoparticle is exposed to air, atmospheric moisture, or room temperature or warmer conditions. Particular embodiments incorporate the stabilizers within each rapamycin nanoparticle, although certain aspects of the invention may be embodied with stabilizers on the surface of the encapsulated rapamycin nanoparticles or otherwise dispersed in the controlled release matrix. To different levels depending on the particular approach used for producing the nanoparticles, with or without other additives, the result is more efficacious for treatment and prevention of genetically-predisposed disorders and age-related disorders, especially in the fields of oncology and neurology in humans and other animals.

Rapid anti-solvent precipitation or solidification, or controlled precipitation, is one method of preparing the rapamycin nanoparticles as it provides for minimal manipulation of the rapamycin and exquisite control over nanoparticle size and distribution, and the crystallinity of the rapamycin. Several controlled precipitation methods are known in the art, including rapid solvent exchange and rapid expansion of supercritical solutions, both of which can be implemented in batch or continuous modes, are scalable, and suitable for handling pharmaceutical compounds. Antisolvent solidification is one approach as it provides exquisite control of particle size and distribution, particle morphology, and rapamycin crystallinity. For example, it is possible to prepare nanoparticles with narrow particle size distribution that are amorphous, crystalline, or combinations thereof. Such properties may exhibit additional benefits, by further controlling the biodistribution and bioavailability of rapamycin in specific indications.

Rapamycin nanoparticles prepared by controlled precipitation methods can be stabilized against irreversible aggregation, Ostwald ripening, and/or reduced dispersibility, by control of colloid chemistry, particle surface chemistry and particle morphology. For example, nanoparticles prepared by antisolvent solidification can be stabilized by ionic and non-ionic surfactants that adsorb to nanoparticle surfaces and promote particle colloid stability through either charge repulsion or steric hindrance, respectively. Moreover, stabilizers can affect nanoparticle crystallinity, which may be used to promote different biodistribution and bioavailability in certain indications.

Rapamycin nanoparticles can consist of molecular rapamycin bound by suitable methods to other nanoparticles. Suitable methods of attaching rapamycin to a nanoparticle carrier or substrate may include physical adsorption through hydrogen van der Waals forces or chemisorption through covalent or ionic bonding. Nanoparticle substrates may be either natural or synthetic, and modified to promote specific interactions with rapamycin. Natural nanoparticles include albumin and other proteins, and DNA. Synthetic nanoparticles include organic and inorganic particulates, micelles, liposomes, dendrimers, hyperbranched polymers, and other compounds.

The rapamycin nanoparticles can be processed by any suitable method, such as by milling, high-pressure atomization, or rapid anti-solvent precipitation. Milling is suitable provided care is taken to minimize both rapamycin degradation and particle agglomeration. Rapamycin degradation can be reduced with the aid of cooling or cryogenic processes. Agglomeration due to the increased surface area and concomitant adhesive forces can be reduced by the use of dispersants during the milling process.

In some embodiments, the rapamycin nanoparticles are sized between about 1 nanometer and about 1 micron. In some embodiments, the rapamycin nanoparticles are less than 1 micron diameter. Such smaller particles provide better control of final particle size, improved stability within the particles, and the ability to tune bioavailability by controlling the crystallinity and composition of the rapamycin nanoparticles.

Manufacturing approaches for the encapsulated rapamycin nanoparticle drug delivery structure embodiments of the present invention include creating a solution of the controlled release matrix, with the rapamycin nanoparticles dispersed therein, in appropriate proportion and producing a heterogeneous mixture. The solvent for such mixtures can be a suitable volatile solvent for the controlled release matrix. In some embodiments, the solvent is either a poor solvent or non-solvent for the rapamycin nanoparticles so that when the rapamycin nanoparticles are dispersed into the controlled release matrix solution they remain as discrete nanoparticles. The resulting dispersion of rapamycin nanoparticles in the controlled release matrix solution can then be reduced to a dry particulate powder by a suitable process, thereby resulting in microparticles of a heterogeneous nature comprised of rapamycin nanoparticles randomly distributed in the controlled release matrix. The particulate powder may also be tailored by a suitable process to achieve a desired particle size for subsequent preparation, which may be from about 20 to about 70 microns in diameter.

The rapamycin nanoparticles are microencapsulated with the controlled release matrix using a suitable particle-forming process to form the encapsulated rapamycin nanoparticle. An example of a particle-forming process is spinning disk atomization and drying. For a detailed discussion of the apparatus and method concerning the aforementioned spin disk coating-process, this application incorporates by references US Patent Applications 2011/221337 and 2011/220430, respectively. Alternatively, for example, the encapsulated rapamycin nanoparticles can be prepared by spray drying.

In some embodiments, not all of the rapamycin nanoparticles will be encapsulated within the controlled release matrix. Instead the rapamycin nanoparticles may be enmeshed with the controlled release matrix, with some of the rapamycin nanoparticles wholly contained within the controlled release matrix while another other rapamycin nanoparticles apparent on the surface of the drug delivery structure, constructed in appearance similar to a chocolate chip cookie.

In some embodiments, and depending on the size of the rapamycin nanoparticles, the encapsulated rapamycin nanoparticles are between 10 and 50 microns in diameter, although diameters as large as 75 microns may be suitable.

The controlled release matrix of the encapsulated rapamycin nanoparticles can be selected to provide desired release characteristics of the encapsulated rapamycin nanoparticles. For example, the matrix may be pH sensitive to provide either gastric release or enteric release of the rapamycin. Enteric release of the rapamycin may achieve improved absorption and bioavailability of the rapamycin. Many materials suitable for enteric release are known in the art, including fatty acids, waxes, natural and synthetic polymers, shellac, and other materials. Polymers are a one enteric coating and may include copolymers of methacrylic acid and methyl methacrylate, copolymers of methyl acrylate and methacrylic acid, sodium alginate, polyvinyl acetate phthalate, and various succinate or phthalate derivatives of cellulose and hydroxpropyl methyl cellulose. Synthetic polymers, such as copolymers of methacrylic acid and either methyl acrylate or methyl methacrlate, are good enteric release polymers due the ability to tune the dissolution pH range of these synthetic polymers by adjusting their comonomer compositions. Examples of such pH sensitive polymers are EUDRAGIT® polymers (Evonik Industries, Essen, Germany). Specifically, EUDRAGIT® S-100, a methyl methacrylate and methacrylic acid copolymer with comonomer ratio of 2:1, respectively, has a dissolution pH of about 7.0, thereby making is suitable for enteric release of rapamycin.

The encapsulated rapamycin nanoparticles may be delivered in various physical entities including a pill, tablet, or capsule. The encapsulated rapamycin nanoparticles may be pressed or formed into a pellet-like shape and further encapsulated with a coating, for instance, an enteric coating. In another embodiment, the encapsulated rapamycin nanoparticles may be loaded into a capsule, also further enterically coated.

Various performance enhancing additives can be added to the encapsulated rapamycin nanoparticles. For example, additives that function as free radical scavengers or stabilizers can be added to improve oxidative and storage stability of the encapsulated rapamycin nanoparticles. In some embodiments, free radical scavengers are chosen from the group that consists of glycerol, propylene glycol, and other lower alcohols. Additives alternatively incorporate antioxidants, such as a tocopherol (vitamin E), citric acid, EDTA, α-lipoic acid, or the like.

Methacrylic acid copolymers with methyl acrylate or methyl methacrylate are moderate oxygen barriers. Furthermore, these polymers will exhibit an equilibrium moisture content. Oxygen transport due to residual solvent, moisture or other causes, can lead to degradation of the encapsulated rapamycin nanoparticles. Oxygen barrier materials can be added to the encapsulated rapamycin nanoparticles formulation to improve oxygen barrier properties. Oxygen barrier polymers compatible with the polymers are polyvinyl alcohol (PVA) and gelatin.

In some embodiments, rapamycin nanoparticle inclusions comprise discrete nanoparticles of rapamycin (or analog) heterogeneously dispersed in a controlled release matrix. In certain embodiments, the rapamycin nanoparticles are prepared by a suitable method and may contain additives to promote nanoparticle stability, modify rapamycin crystallinity, or promote compatibility of the rapamycin nanoparticles with the controlled release matrix. The controlled release matrix is formulated to promote release of rapamycin to specific parts of the body, such as the intestine, to enhance oxidative and storage stability of the encapsulated rapamycin nanoparticles, and to maintain the discrete, heterogeneously distributed nature of the rapamycin nanoparticles.

In specific embodiments, rapamycin is dissolved in a suitable water-miscible solvent and then rapidly injected into rapidly stirred water containing an appropriate aqueous soluble dispersant. Water-miscible solvents for rapamycin include methanol, ethanol, isopropyl alcohol, acetone, dimethylsulfoxide, dimethylacetamide, n-methylpyrolidone, tetrahydrofuran, and other solvents. Low boiling point, high vapor pressure water-miscible solvents facilitate their removal during subsequent microparticle formation. Examplary water-miscible solvents are methanol, acetone, and isopropyl alcohol. In some embodiments, the water-miscible solvent is methanol. Some aqueous soluble dispersants include ionic surfactants such as sodium dodecyl sulfate and sodium cholate, non-ionic surfactants such as Pluronics, Poloxomers, Tweens, and polymers, such as polyvinyl alcohol and polyvinylpyrolidone. Examplary aqueous-soluble dispersants are sodium cholate, Pluronic F-68, and Pluronic F-127. In some embodiments, the aqueous-soluble dispersant is sodium cholate, which provides surprisingly beneficial properties. Not only is sodium cholate a surfactant and a dispersant, it serves to cause aggregation of rapamycin particles from the aqueous solution. Moreover, while sodium cholate tends to be a polar molecule as well as an amphoteric surfactant, it surrounds each nanoparticle with a hydrophobic charge when it is enmeshed in the Eudragit matrix. Then, when the nanoparticle is released from the Eudragit matrix within the animal subject's enteric passages where conditions are basic, the same properties cause the nanoparticle to be more readily received and absorbed through the intestinal walls.

In certain embodiments, rapamycin is dissolved in the water-miscible solvent at a concentration of about 0.01% w/v to about 10.0% w/v preferably about 0.1% w/v to about 1.0% w/v. The aqueous-soluble dispersant is dissolved in water at a concentration above its critical micelle concentration, or CMC, typically at about 1 to about 10 times the CMC. The rapamycin solution is injected into the aqueous-soluble dispersant solution with agitation at a volumetric ratio of about 1:10 to about 1:1, preferably about 1:5 to about 1:1.

The controlled release matrix is prepared from a water-soluble polymer, which may be a copolymer of methacrylic acid with either methyl acrylate or methyl methacrylate, such as those marketed under the trade name of EUDRAGIT® and having pH-dependent dissolution properties. The controlled release matrix may be comprised of EUDRAGIT® S-100, although other water-soluble enteric controlled release would be suitable. Water-soluble controlled release matrices are selected so as either not to compromise the integrity of rapamcyin nanoparticles or to provide a medium in which rapamycin nanoparticles may be prepared by the controlled precipitation methodology described previously.

In preparing the water-soluble polymer it is helpful to maintain conditions that do not compromise the integrity of the rapamycin nanoparticles. Firstly, since the rapamycin nanoparticles are susceptible solubilization by certain co-solvents, it is important to maintain a suitable quantity of certain co-solvents to achieve controlled release matrix solubility while not deleteriously affecting the morphology of the rapamycin nanoparticles. Secondly, rapamycin nanoparticles will be susceptible to chemical degradation by high pH; therefore, it is important to modulate the controlled release matrix solution pH so that rapamycin is not chemically altered. It is helpful the controlled release matrix solution pH be maintained below about pH 8. Lastly, it is helpful to achieve near to complete solubilization of the controlled release matrix in solution so that microencapsulation of the rapamycin nanoparticles by the controlled release matrix in subsequent processing steps may proceed with high efficiency. When using the EUDRAGIT® S-100 as the controlled release matrix, it is helpful to achieve a controlled release matrix solution by using a combination of co-solvents and solution pH modulation. In certain embodiments, the co-solvents are about 40% or less by volume. Similarly, in certain embodiments, the pH of the controlled release matrix solution is about 8 or less, such that the EUDRAGIT® S-100 is not completely neutralized and may be only about 80% or less neutralized. These conditions achieve nearly complete to complete solubilization of the EUDRAGIT® S-100 in a medium that is mostly aqueous and that maintains the integrity of the rapamycin nanoparticles, therefore leading to their microencapsulation by the controlled-release matrix in subsequent processing steps.

The rapamycin nanoparticles prepared by the controlled precipitation method are added to the aqueous solution of the controlled released matrix, resulting in a nanoparticle dispersion in the solubilized controlled release matrix. Alternatively, the rapamycin solubilized in a suitable co-solvent can be dispersed into the aqueous solution of controlled release matrix leading to controlled precipitation of rapamycin particles, thereby leading to a rapamycin nanoparticle dispersion in fewer processing steps, but of appropriate composition to permit subsequent microencapsulation processing.

As an alternative embodiment, the encapsulated rapamycin nanoparticles are created using pre-existing nanoparticle substrates, such as albumin, to create, in the case of albumin, “albumin-rapamycin nanoparticles.” Within this general class of alternatives, certain approaches for creating the albumin-rapamycin nanoparticles involve encapsulating rapamycin within albumin nanoparticles or preferentially associating rapamycin with albumin nanoparticles through physical or chemical adsorption. The albumin nanoparticles themselves may be formed from human serum albumin, a plasma protein derived from human serum.

More particularly, this embodiment may involve use of a therapeutic peptide or protein that is covalently or physically bound to albumin, to enhance its stability and half-life. With the albumin stabilized, the rapamycin is mixed with the stabilized albumin in an aqueous solvent and passed under high pressure to form rapamycin-albumin nanoparticles in the size range of 100-200 nm (comparable to the size of small liposomes).

Certain embodiments also address degradation risks and other limits imposed by the related art by preparing encapsulated rapamycin nanoparticles as a heterogeneous mixture of rapamycin nanoparticles in a polymer matrix. Distributed nanoparticles are morphologically different than homogeneous rapamycin; and are less susceptible to degradation because of the bulk nature of the nanoparticles compared to the smaller size of molecular rapamycin.

V. Biodegradable Polymers Loaded with Rapamycin

In some aspects, the compositions of the present invention comprise biodegradable polymers loaded with rapamycin. Biodegradable polymers loaded with drugs can be microparticles. “Microparticle” refers to particles between about 0.1 and 300 μm in size. Drug-loaded biodegradable polymers release drug in a time-dependent manner.

As used herein, “biodegradable” refers to any natural means by which a polymer can be disposed of in a patient's body. This includes such phenomena as, without limitation, biological decomposition, bioerosion, absorption, resorption, etc. Biodegradation of a polymer in vivo results from the action of one or more endogenous biological agents and/or conditions such as, without limitation, enzymes, microbes, cellular components, physiological pH, temperature and the like.

In some aspects, the biodegradable polymers can be poly-ε-caprolactone (PCL) microparticles. PCL is a biodegradable, biocompatible, and semicrystalline polymer. PCL is useful for controlled drug delivery because it is highly permeable to many drugs and is non-toxic. Sinha et al. 2004. Rapamycin can also loaded onto microparticles of other biodegradable polymers, including but not limited to aliphatic polyesters, polylactide, polyglycolide, poly(lactide-co-glycolide), mixtures thereof, and their copolymers. Such biodegradable polymers are known in the art.

Rapamycin may be loaded onto microspheres of PCL alone or of PCL copolymers or blends to obtain the desired drug release characteristics. Copolymers of PCL can be formed using many different monomers, including but not limited to ethyleneoxide, polyvinylchloride, chloroprene, polyethylene glycol, polystyrene, diisocyanates (urethanes), tetrahydrofuran (THF), diglycolide, dilactide, δ-valerlactone, substituted caprolactones, 4-vinyl anisole, styrene, methyl methacrylate, and vinyl acetate.

In some aspects, colon targeting of rapamycin can be achieved by creating PCL microparticles loaded with rapamycin or rapamycin analog and subsequently coating the microparticles with Eudragit S 100. Methods of making such coated microparticles can be found in Ghorab et al., 2011, which is hereby incorporated by reference. Briefly, drug-loaded PCL microparticles are suspended in a solution containing an appropriate amount of Eudragit S 100 disolved in ethyl alcohol. The suspension is poured into distilled water. The resulting mixture is homogenized for five minutes and then mechanically stirred until the organic solvent is completely evaporated. Microparticles are collected, washed with cyclohexane twice, and dried overnight in a dessicator.

Drug-loaded PCL microspheres can be prepared by several different methods known by persons of skill in the art, including but not limited to an o/w emulsion solvent extraction/evaporation method, a w/o/w emulsion solvent evaporation technique, a spray drying technique, a solution-enhanced dispersion method, and a hot melt technique. These methods are described in more detail in Sinha et al., 2004, which is hereby incorporated by reference. Briefly, as a non-limiting example, the o/w emulsion solvent extraction evaporation method can be performed by dissolving the required amount of polymer and drug in an organic phase, emulsifying under stirring with polyvinyl alcohol to form an o/w emulsion, stirring for 3 hours at about 500 rpm to evaporate the organic phase, and filtering and drying the formed microspheres.

Drug-loaded microspheres of aliphatic polyesters, polylactide, polyglycolide, and poly(lactide-co-glycolide) can be prepared by several different methods known by persons of skill in the art. Non-limiting examples can be found in the following references, all of which are hereby incorporated by reference: Kemala et al., 2012; Ghassemi et al., 2009; Corrigan & Heelan, 2001; Cleland et al., WIPO Pub. No. WO 1995/11009; and Atkins et al., WIPO Pub. No. WO 1995/009613.

### VI. Pharmaceutical Preparations

Certain methods and compositions set forth herein are directed to administration of an effective amount of a composition comprising the mTOR inhibitor (including rapamycin and/or rapamycin analog) compositions of the present disclosure.

A. Compositions

A “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (Remington's, 1990). Except insofar as any conventional carrier is incompatible with the active ingredient, its use in the therapeutic or pharmaceutical compositions is contemplated. The compositions used in the present invention may comprise different types of carriers depending on whether it is to be administered in solid, liquid or aerosol form, and whether it needs to be sterile for such routes of administration as injection.

The use of such media and agents for pharmaceutically active substances is well known in the art. Except insofar as any conventional media or agent is incompatible with the active ingredient, its use in the therapeutic compositions is contemplated. Supplementary active ingredients can also be incorporated into the compositions, and these are discussed in greater detail below. For human administration, preparations should meet sterility, pyrogenicity, general safety and purity standards as required by FDA Office of Biologics standards.

The formulation of the composition may vary depending upon the route of administration. For parenteral administration in an aqueous solution, for example, the solution should be suitably buffered if necessary and the liquid diluent first rendered isotonic with sufficient saline or glucose. In this connection, sterile aqueous media that can be employed will be known to those of skill in the art in light of the present disclosure.

In addition to the compounds formulated for parenteral administration, such as intravenous or intramuscular injection, other pharmaceutically acceptable forms include, e.g., tablets or other solids for oral administration; liposomal and nanoparticle formulations; enteric coating formulations; time release capsules; formulations for administration via an implantable drug delivery device, and any other form. One may also use nasal solutions or sprays, aerosols or inhalants in the present invention.

In embodiments wherein capsules are utilized, the capsules may be, for example, hard shell capsules or soft-shell capsules. The capsules may optionally include one or more additional components that provide for sustained release.

In certain embodiments, pharmaceutical composition includes at least about 0.1% by weight of the active compound. In other embodiments, the pharmaceutical composition includes about 2% to about 75% of the weight of the composition, or between about 25% to about 60% by weight of the composition, for example, and any range derivable therein.

The compositions may comprise various antioxidants to retard oxidation of one or more components. Additionally, the prevention of the action of microorganisms can be accomplished by preservatives such as various antibacterial and antifungal agents, including but not limited to parabens (e.g., methylparabens, propylparabens), chlorobutanol, phenol, sorbic acid, thimerosal or combinations thereof. The composition should be stable under the conditions of manufacture and storage, and preserved against the contaminating action of microorganisms, such as bacteria and fungi.

In certain embodiments, an oral composition may comprise one or more binders, excipients, disintegration agents, lubricants, flavoring agents, and combinations thereof. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

In particular embodiments, prolonged absorption can be brought about by the use in the compositions of agents delaying absorption, such as, for example, aluminum monostearate, gelatin, or combinations thereof.

B. Routes of Administration

Compositions may be administered in a manner compatible with the dosage formulation and in such amount as is therapeutically effective. In specific embodiments, the composition is encased, encapsulated, or coated.

The composition can be administered to the subject using any method known to those of ordinary skill in the art. For example, a pharmaceutically effective amount of the composition may be administered intravenously, intracerebrally, intracranially, intraventricularly, intrathecally, into the cortex, thalamus, hypothalamus, hippocampus, basal ganglia, substantia nigra or the region of the substantia nigra, cerebellum, intradermally, intraarterially, intraperitoneally, intralesionally, intratracheally, intranasally, topically, intramuscularly, anally, subcutaneously, orally, topically, locally, inhalation (e.g., aerosol inhalation), injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art (Remington's, 1990).

In particular embodiments, the composition is administered to a subject using a drug delivery device. Any drug delivery device is contemplated for use in delivering an effective amount of the inhibitor of mTOR.

C. Dosage

A pharmaceutically effective amount of an inhibitor of mTOR is determined based on the intended goal. The quantity to be administered, both according to number of treatments and dose, depends on the subject to be treated, the state of the subject, the protection desired, and the route of administration. Precise amounts of the therapeutic agent also depend on the judgment of the practitioner and are peculiar to each individual.

The amount of rapamycin or rapamycin analog or derivative to be administered will depend upon the disease to be treated, the length of duration desired and the bioavailability profile of the implant, and the site of administration. Generally, the effective amount will be within the discretion and wisdom of the patient's physician. Guidelines for administration include dose ranges of from about 0.01 mg to about 500 mg of rapamycin or rapamycin analog.

For example, a dose of the inhibitor of mTOR may be about 0.0001 milligrams to about 1.0 milligrams, or about 0.001 milligrams to about 0.1 milligrams, or about 0.1 milligrams to about 1.0 milligrams, or even about 10 milligrams per dose or so. Multiple doses can also be administered. In some embodiments, a dose is at least about 0.0001 milligrams. In further embodiments, a dose is at least about 0.001 milligrams. In still further embodiments, a dose is at least 0.01 milligrams. In still further embodiments, a dose is at least about 0.1 milligrams. In more particular embodiments, a dose may be at least 1.0 milligrams. In even more particular embodiments, a dose may be at least 10 milligrams. In further embodiments, a dose is at least 100 milligrams or higher.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

The dose can be repeated as needed as determined by those of ordinary skill in the art. Thus, in some embodiments of the methods set forth herein, a single dose is contemplated. In other embodiments, two or more doses are contemplated. In some embodiments, the two or more doses are the same dosage. In some embodiments, the two or more doses are different dosages. Where more than one dose is administered to a subject, the time interval between doses can be any time interval as determined by those of ordinary skill in the art. For example, the time interval between doses may be about 1 hour to about 2 hours, about 2 hours to about 6 hours, about 6 hours to about 10 hours, about 10 hours to about 24 hours, about 1 day to about 2 days, about 1 week to about 2 weeks, or longer, or any time interval derivable within any of these recited ranges. In specific embodiments, the composition may be administered daily, weekly, monthly, annually, or any range therein.

In certain embodiments, it may be desirable to provide a continuous supply of a pharmaceutical composition to the patient. This could be accomplished by catheterization, followed by continuous administration of the therapeutic agent. The administration could be intra-operative or post-operative.

Doses for embodiments of encapsulated rapamycin (eRapa) and for encapsulated rapamycin nanoparticles maybe different. According to certain embodiments, doses are contemplated in a range of more than 50 micrograms and up to (or even exceeding) 200 micrograms per kilogram for daily administration, or the equivalent for other frequencies of administration. Although dosing may vary based on particular needs and preferred treatment protocols according to physician preference, maximum tolerable daily bioavailable dosings (trough levels) for a 28-day duration are about 200 micrograms of rapamycin (or equivalent) per subject kilogram, for both human and canine subjects, although those of ordinary skill would understand that greater dose amount ranges would be tolerable and suitable when administered less often than once per day, and lesser ranges would be tolerable when administered more often than once per day.

### VII. Kits

Kits are also contemplated as being used in certain aspects of the present invention. For instance, one or more mTOR inhibitors (including rapamycin and/or rapamycin analogs) composition of the present invention can be included in a kit. A kit can include a container. Containers can include a bottle, a metal tube, a laminate tube, a plastic tube, a dispenser, a pressurized container, a barrier container, a package, a compartment, or other types of containers such as injection or blow-molded plastic containers into which the compositions are retained. The kit can include indicia on its surface. The indicia, for example, can be a word, a phrase, an abbreviation, a picture, or a symbol.

The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the mTOR inhibitor composition and any other reagent containers in close confinement for commercial sale.

However, the components of the kit may be provided as dried powder(s). When reagents and/or components are provided as a dry powder, the powder can be reconstituted by the addition of a suitable solvent. It is envisioned that the solvent may also be provided in another container means.

Further, the rapamycin compositions of the present invention may also be sterile, and the kits containing such compositions can be used to preserve the sterility. The compositions may be sterilized via an aseptic manufacturing process or sterilized after packaging by methods known in the art.

In certain aspects, the kit comprises instructions for a user. The instructions direct the user to administer the one or more mTOR inhibitors (including rapamycin and/or rapamycin analogs) to the individual no sooner than at least 2, 3, or 4 or more days following a procedure.

### EXAMPLES

The following examples are included to demonstrate certain non-limiting aspects of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention.

### Example 1

In the course of investigations into the effects of blockade of the mammalian target of rapamycin (mTOR) by rapamycin in vivo in middle-aged human volunteers, 3 mm skin biopsies were performed: one at a site that received rapamycin for a week; and a second at a nearly site that was treated with the ointment vehicle only. During a post-biopsy visit with a keloid-susceptible volunteer subject, it was noted that the biopsy site that received vehicle only healed with a small keloid, while the site that received rapamycin ointment healed without any lesion whatsoever. This observation suggested that rapamycin ointment might prevent keloid formation. Indeed, the differences in keloid formation at biopsy sites were replicated in the keloid-susceptible volunteer during a second trial. In the course of delivering clinical care, it was noted that there were 2 patients who suffered from clinically significant intra-abdominal adhesions who also developed keloids. Thus, in specific embodiments, there is a relationship between the problem these patients had with adhesions and that they both developed keloids. Keloids are similar to intra-abdominal adhesions in that they do not regress spontaneously and they tend to recur after excision. Furthermore, keloids are histologically similar to intra-abdominal adhesions. In both lesions, there is excess production and deposition of extracellular matrix, especially collagen and fibronectin. Compared to normal tissue, both express a number of genes that regulate cell growth and apoptosis, inflammation, angiogenesis, and tissue turnover. Epidemiological work found that women with keloids had more intra-abdominal adhesion between the uterus and bladder and between the uterus and anterior abdominal wall than those without keloids (Tulandi et al., 2011). Furthermore, keloids are histologically similar to intra-abdominal adhesions. In both lesions there is excess production and deposition of extracellular matrix, especially collagen and fibronectin. Compared to normal tissue, both express a number of genes that regulate cell growth and apoptosis, inflammation, angiogenesis, and tissue turnover. It was considered that if topical application of rapamycin prevented the formation of new keloid scarring, that in specific embodiments systemic treatment with rapamycin will prevent post-surgical abdominal adhesion formation and in certain embodiments regress already extant adhesions. Because rapamycin can also retard wound healing, however, initiation of rapamycin anti-adhesion therapy should be delayed following a medical procedure, such as at least 2, 3, or 4 or more days to allow surgical wound healing to begin without mTOR inhibition. After that period, mTOR inhibitor therapy can be initiated.

There is no research showing that rapamycin alters adhesion formation or regression. Indeed, Dietrich et al (2012) concluded that rapamycin was of no benefit in preventing adhesions when injected into the peritoneal cavity beginning immediately post-surgery.

Delaying initiation of rapamycin therapy for 2, 3, or 4 or more days from the time of surgery will allow normal wound healing to begin, but yet still prevent clinically significant adhesion formation. Oral treatment with mTOR antagonists, especially with a formulation that is released in the lower intestine, for example, can avoid trauma associated with repeated injections and deliver maximal drug to the peritoneal cavity where adhesions form. Finally, in particular embodiments treatment with oral rapamycin or other mTOR antagonists will regress established adhesions, which now are only treated with surgery and usually recur.

The use of rapamycin to regress established adhesions or prevent new adhesion formation provides a novel therapy for a health problem for which no satisfactory treatment exists.

### Example 2

In the course of delivering clinical care, it was noticed that 2 patients who suffered from clinically significant intra-abdominal adhesions also developed keloids. At that same time, the inventor was utilizing topical RAPA ointment in studies and found that it prevented and regressed keloids. It was considered noteworthy that keloids are similar to intra-abdominal adhesions in that they do not regress spontaneously and they tend to recur after excision. Furthermore, keloids are histologically similar to intra-abdominal adhesions. In both lesions there is excess production and deposition of extracellular matrix, especially collagen and fibronectin. Compared to normal tissue, both express a number of genes that regulate cell growth and apoptosis, inflammation, angiogenesis, and tissue turnover. Initial studies indicated that topical application of rapamycin prevented the formation of new keloid scarring and, in fact, appeared to cause regression of existing keloid scars. It was considered that systemic treatment with rapamycin would prevent post-surgical abdominal adhesion formation as well as regress existing adhesions. This consideration can be tested, for example, with RAPA-fed Sprague-Dawley rats. For example, one can determine whether systemic rapamycin administration prevents intra-abdominal adhesion formation in a post-surgical adhesion model in rats. Intra-abdominal adhesions can be induced in four groups of rats—two control groups and two groups to be fed rapamycin-laced chow for either 5 days or 16 days. Rats can be sacrificed and adhesions graded by both macroscopic and microscopic observation. One can also determine whether systemic rapamycin administration regresses established intraabdominal adhesions in a post-surgical adhesion model in rats. Intra-abdominal adhesions can be induced in rats, which can be allowed to heal for three weeks, a time period sufficient to complete adhesion formation. Subsequently, one group can be fed rapamycin-laced chow, and the other can receive control chow. After three weeks (six weeks past surgery), rats are sacrificed and adhesions graded by both macroscopic and microscopic observation.

Initial studies indicated that topical application of rapamycin (RAPA) prevented the formation of new keloid scarring and, in fact, appeared to cause regression of existing keloid scars. These observations led to the consideration that systemic treatment with rapamycin would prevent post-surgical abdominal adhesion formation as well as regress existing adhesions. This consideration can be tested with RAPA-fed Sprague-Dawley rats.

This disclosure addresses all of these considerations: 1) the enterically-delivered RAPA protocol eliminates the potential side effects caused by repeated IP injection, thus obviating the confounding effects of repeated trauma as in the work by Dietrich et al. (2012); 2) one can follow adhesion formation at both early and late stages, unlike Dietrich et al. (2012); 3) one can use an adhesion grading system based on both gross and microscopic examinations, and one can modify the grading system if required; and 4) delaying RAPA administration until wound healing that has progressed for 4 days (as an example of a period of time for delay) eliminates the side effects of weakened wound healing.

Initial Studies

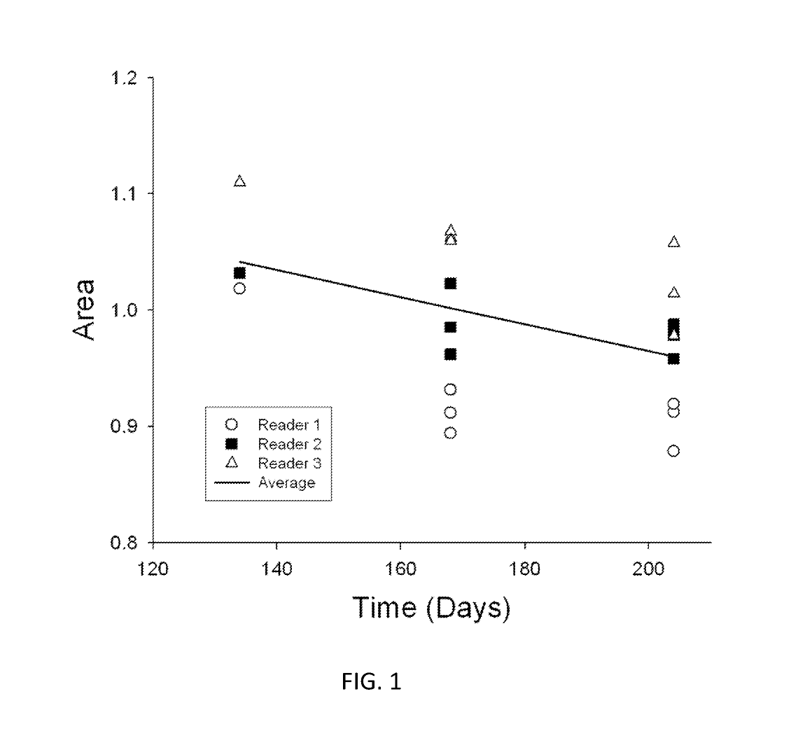

Rapamycin, Keloids, and Adhesions: As previously mentioned, there were 2 adhesion/keloid patients at a time when the inventor was performing investigations into the effects of blockade of the mammalian target of rapamycin (mTOR) by RAPA in vivo in middle-aged human volunteers. As part of this study, 3 mm skin punch biopsies were performed, one at a site that received RAPA for a week and a second at a nearly site that was treated with the ointment vehicle only. During a postbiopsy visit with a keloid-susceptible volunteer subject, it was noted that the biopsy site that received vehicle only healed with a small keloid while the site that received 8% RAPA ointment healed without any lesion whatsoever. This serendipitous observation suggested that 8% RAPA ointment might prevent keloid formation. Indeed, the differences in keloid formation at biopsy sites were replicated in the keloid susceptible volunteer during a second trial. Furthermore, reports that RAPA increases collagenase indicated that topical RAPA might also regress established keloids (Poulalhon et al., 2006). To explore this possibility, the keloid-susceptible volunteer applied RAPA ointment to an established keloid twice per day. Digital photographs with metric rulers adjacent to the keloid were taken to objectively monitor changes in lesion size. Surface area calculations from the digital photos were calculated by image analysis with Image J. This program can measure surface areas based on pixel analyses when calibrated against a standard; in this case the standard was a millimeter ruler in the photograph, adjacent to the keloid. After 2 months of treatment the volunteer stated that he believed the lesion was getting smaller, although this was not apparent from lesion surface area calculations. (In retrospect, it is considered that in the first months of treatment the area did not decrease but the height of the lesion above the surrounding skin decreased). After 4 months, the keloid surface area calculated from the photographs taken under identical lighting and distance conditions showed a tendency toward reduction. After 7 months, surface area calculations made by 3 different blinded observers with Image J showed a clear reduction in surface area (FIG. 1; calculated surface area in cm² with linear regression lines for months 4-7). The plot shows the lesion area measured by three readers from multiple photographs vs. follow-up time. The data were analyzed using analysis of variance (ANOVA) for repeated measurements (Winer, 1971).

The statistical model included the effect of follow-up time, a random effect of photograph within follow-up time, a random effect of reader, an interaction of time and reader, an interaction of reader and photograph, and a residual error. The largest variance component was due to reader (variance component estimate 0.0036) with the residual error being the second largest (0.0011); estimates of the other variance components were substantially smaller (photograph within time 0.0002, reader by time 0.0002, and reader by photograph (0.0001). There was a significant linear trend (slope=−0.0013, P=0.0163); the quadratic trend was not statistically significant (P=0.2728). The negative linear trend was observed for each reader (Reader 1 −0.0016, P=0.0066; Reader 2 −0.0008, P=0.1118; Reader 3 −0.0013, P=0.0459). This exciting observation led us to propose a pilot study to NIAMS to test topical application of RAPA ointment to regress keloids.

Exemplary Approaches

Approach 1. Determine whether systemic rapamycin administration prevents intra-abdominal adhesion formation in a post-surgical adhesion model in rats. Rationale: Topical treatment with RAPA appears to prevent keloid scar formation. Since keloid and adhesion tissue have similar compositions and potentially similar pathways of formation, it is reasonable to hypothesize that RAPA will have the same preventative effect on adhesions as on keloids.

A total of 40 male Sprague-Dawley rats (20 eRAPA-treated, 20 control), aged 16-20 weeks, can undergo midline laparotomy as previously described (Dietrich et al., 2012). Before the surgery, rats can be anesthetized with a single intramuscular injection of 100 mg/kg ketamine, 10 mg/kg xylazine, and 0.2 mg/kg atropine. Rats can be placed in dorsal recumbency, and a midline laparotomy can be performed. The cecum can be externalized, and serosa of 2 cm² can be abraded by scratching using anatomical forceps until serosal bleeding occurs. The abdominal wall can be closed with three continuous layers, including intradermal skin sutures.

Novalminsulfonium (100 mg/kg) can be given for 3 days postoperatively via drinking water. On day 4 postsurgery, rats can be switched to chow containing microencapsulated rapamycin (eRAPA, a novel formulation of enterically delivered rapamycin; 14 mg/kg food designed to deliver ˜2.24 mg of rapamycin per kg body weight/day to achieve about 4 ng/ml of rapamycin per kg body weight/day) prepared by TestDiet, Inc., Richmond, Ind. using Purina 5LG6 as the base. Control diet was the same but with empty capsules. This feeding protocol has been used in initial rat studies at the Barshop and has demonstrated efficacy in delivering systemic levels of RAPA. One can monitor the blood concentration of eRAPA weekly throughout the experimental protocol via HPLC-tandem mass spectrometry, as described (Livi et al., 2013). One can measure mTOR activity in intestinal tissue (post-sacrifice) by immunoblot analysis of mTOR-mediated phosphorylation of ribosomal protein S6 kinase, which should be decreased in the RAPA-treated animals.

Twenty of the animals (10 RAPA-treated and 10 controls) can be sacrificed on day 9 (surgery is day 1) and 20 animals (10 RAPA-treated and 10 controls) can be sacrificed on day 21. Day 9 represents an early stage of adhesion formation, when collagen deposition begins and before granulation becomes fibrosis. Day 21 represents a later, final stage in adhesion development, where adhesion formation is complete. Adhesions can be quantified using a scoring system described by Moreno et al. (1996). The degree of peritoneal adhesion formation can be assessed based on the combined parameters of macroscopic and microscopic observations. The score can be calculated based on the number of adhesions, the site of adhesions, presence of vascularization, thickness, and strength (Moreno et al., 1996). Plasma levels of tissue-type plasminogen activator (tPA), and plasminogen activator inhibitor-1 (PAI-1) will be measured by ELISA (AbCam). tPA activates plasminogen, which is processed into plasmin, which then degrades fibrin, and so attenuates adhesion formation. PAI-1 prevents activation of plasminogen and so promotes accumulation of fibrin and, thus, adhesion formation.

In specific embodiments, the eRAPA-fed animals can exhibit fewer and milder adhesions as compared to the control group, which can form more, relatively severe adhesions. Furthermore, the RAPA-treated animals exhibit higher levels of plasma tPA, but lower levels of plasma PAI-1, than the control animals, in certain embodiments. A particular method for the induction of intra-abdominal adhesions is chosen; there are, however, other accepted methods as described in Gaertner et al. (2008) and Ozel et al. (2005). If necessary, one can use an alternative method to generate adhesions.

Approach 2. Determine whether systemic rapamycin administration regresses established intra-abdominal adhesions in a post-surgical adhesion model in rats. Rationale: Topical treatment with RAPA was shown to regress established keloid scar tissue. Since keloid and adhesion tissue have similar compositions and potentially similar pathways of formation, it is reasonable to propose that RAPA treatment of established adhesion scar tissue will undergo comparable regression.

Twenty male Sprague-Dawley rats (10 eRAPA-treated, 10 control), aged 16-20 weeks, can undergo midline laparotomy. The rats can be allowed to heal for 3 weeks post-surgery, allowing for adhesion formation, all eating the control chow. The RAPA-treatment group can then be switched to eRAPA-containing chow, on which they can remain for 3 weeks. As in Approach 1, RAPA plasma levels can be evaluated weekly. At the end of the prescribed time, animals can be sacrificed and their abdominal adhesions graded as described in Approach 1. Plasma levels of tissue-type plasminogen activator (tPA), and plasminogen activator inhibitor-1 (PAI-1) can also be assessed.

In specific embodiments, RAPA regresses existing adhesions, and the eRAPA-fed animals exhibit milder adhesions as compared to the control group, which should present with significant adhesions. Furthermore, in certain embodiments the RAPA-treated animals exhibit higher levels of plasma tPA, but lower levels of plasma PAI-1, than the control animals.

### REFERENCES

Arung, W., Meurisse, M. and Detry, O. (2011) Pathophysiology and prevention of postoperative peritoneal adhesions. World journal of gastroenterology: WJG 17, 4545.

Dietrich A, Bouzidi M, Hartwig T, Schutz A, and Jonas S. Rapamycin and a hyaluronic acidcarboxymethylcellulose membrane did not lead to reduced adhesion formations in a rat abdominal adhesion model. Arch Gyneco/Obstet 285: 1603-1609, 2012.

Gaertner, W. B., Hagerman, G. F., Felemovicius, I., Bonsack, M. E. and Delaney, J. P. (2008) Two experimental models for generating abdominal adhesions. J Surg Res 146, 241-245.

Hellebrekers, B. W. J. and Kooistra, T. (2011) Pathogenesis of postoperative adhesion formation. British Journal of Surgery 98, 1503-1516.

Livi, C. B., Hardman, R. L., Christy, B. A., Dodds, S. G., Jones, D., Williams, C., Strong, R., Bokov, A., Javors, M. A. and Ikeno, Y. (2013) Rapamycin extends life span of Rb1+/− mice by inhibiting neuroendocrine tumors. Aging (Albany N.Y.) 5, 100.

Moreno, A., Aguayo, J. L., Zambudio, G., Ramirez, P., Canteras, M. and Parrilla, P. (1996) Influence of abdominal incision on the formation of postoperative peritoneal adhesions: an experimental study in rats. Eur J Surg 162, 181-185.

Ozel, H., Avsar, F. M., Topaloglu, S. and Sahin, M. (2005) Induction and assessment methods used in experimental adhesion studies. Wound Repair Regen 13, 358-364.

Poulalhon, N., Farge, D., Roos, N., Tacheau, C., Neuzillet, C., Michel, L., Mauviel, A. and Verrecchia, F. (2006) Modulation of Collagen and MMP-1 Gene Expression in Fibroblasts by the Immunosuppressive Drug Rapamycin A DIRECT ROLE AS AN ANTIFIBROTIC AGENT? Journal of biological chemistry 281, 33045-33052.

Tulandi T, AI-Sannan B, Akbar G, Ziegler C, and Miner L. Prospective study of intraabdominal adhesions among women of different races with or without keloids. Am J Obstet Gyneco/204: 132.e131-134, 2011.

Tulandi, T. and Lyell, D. J. (2013) Classification of intra-abdominal adhesions after cesarean delivery. Gynecological Surgery 10, 25-29.

Ward, B. C. and Panitch, A. (2011) Abdominal adhesions: current and novel therapies. J Surg Res 165, 91-111.

Willems, M. C., Hendriks, T., de Man, B. M., Lomme, R. M. and van der Vliet, J. A. (2011) Everolimus-induced loss of wound strength can be prevented by a short postoperative delay in its administration. Wound Repair Regen 19, 680-686.

Winer, B. J. (1971) Statistical principles in experimental design. New York, McGraw-Hill.

What is claimed is:

1. A method of
a) preventing or reducing adhesion between two tissues and/or organs in an individual subjected to a procedure, and/or
b) preventing or reducing one or more keloids in an individual subjected to a procedure;
comprising the step of providing to the individual an effective amount of a composition comprising one or more inhibitors of an mTOR pathway no earlier than about 4 days following the procedure.

2. The method of claim 1, wherein the inhibitor of an mTOR pathway is rapamycin and/or a rapamycin analog.

3. The method of claim 2, wherein the rapamycin analog is selected from the group consisting of temsirolimus, everolimus, deforolimus, CCI-779, curcumin, Green tea extract standardised to 70% EGCG, transresveratrol, fisetin, salicin extracted from white willow, and a combination thereof.

4. The method of claim 1, wherein the inhibitor of the mTOR pathway is an ATP-competitive mTOR kinase inhibitor.

5. The method of claim 4, wherein the ATP-competitive mTOR kinase inhibitor is selected from the group consisting of AZD8055, Torin1, PP242, PP30 and a combination thereof.

6. The method of any of claims 1-5, wherein the adhesions are between abdominal, pelvic, or thoracic organs and/or with the walls of the abdominal, pelvic, or thoracic cavities.

7. The method of claim 6, wherein the pelvic adhesions involve a reproductive organ, the urinary bladder, the pelvic colon, and/or the rectum.

8. The method of claim 6, wherein the abdominal adhesions involve the stomach, liver, gallbladder, spleen, pancreas, small intestine, kidney, large intestine, and/or adrenal gland.

9. The method of any of claims 1-8, wherein the procedure is an abdominal, thoracic or gynecological surgery.

10. The method of any of claim 2-3 or 6-10, wherein the rapamycin or rapamycin analog is encased in a coating that comprises a cellulose acetate succinate or hydroxy propyl methyl cellulose phthalate co-polymer, or a polymethacrylate-based copolymer to include: methyl acrylate-methacrylic acid copolymer, or a methyl methacrylate-methacrylic acid copolymer.

11. The method of claim 11, wherein the coating comprises Poly(methacylic acid-co-ethyl acrylate) in a 1:1 ratio, Poly(methacrylic acid-co-ethyl acrylate) in a 1:1 ratio, Poly(methacylic acid-co-methyl methacrylate) in a 1:1 ratio, Poly(methacylic acid-co-methyl methacrylate) in a 1:2 ratio, Poly(methyl acrylate-co-methyl methacrylate-co-methacrylic acid) in a 7:3:1 ratio, Poly(ethyl acrylate-co-methyl methacrylate-co-trimethylammonioethyl methacrylate chloride) in a 1:2:0.2 ratio, Poly(ethyl acrylate-co-methyl methacrylate-co-trimethylammonioethyl methacrylate chloride) in a 1:2:0.1 ratio, or Poly(butyl methacylate-co-(2-dimethylaminoethyl) methacrylate-co-methyl methacrylate) in a 1:2:1 ratio, a naturally-derived polymer, or a synthetic polymer, or any combination thereof.

12. The method of claim 12, wherein the naturally-derived polymer is selected from the group consisting of alginates and their various derivatives, chitosans and their various derivatives, carrageenans and their various analogues, celluloses, gums, gelatins, pectins, and gellans.

13. The method of claim 12, wherein the naturally-derived polymer is selected from the group consisting of polyethyleneglycols (PEGs) and polyethyleneoxides (PEOs), acrylic acid homo- and copolymers with acrylates and methacrylates, homopolymers of acrylates and methacrylates, polyvinyl alcohol PVOH), and polyvinyl pyrrolidone (PVP).

14. The method of any one of claims 1-14, wherein the inhibitor is provided to the individual topically.

15. The method of any one of claims 1-14, wherein the inhibitor is provided to the individual systemically.

16. The method of any of claims 1-14, wherein the composition is administered orally or enterically.

17. The method of any of claim 2-3 or 6-17, wherein the composition comprises rapamycin or a rapamycin analog at a concentration of 0.001 mg to 30 mg total per dose.

18. The method of any of claim 2-3 or 6-17, wherein the composition comprising rapamycin or an analog of rapamycin comprises 0.001% to 60% by weight of rapamycin or an analog of rapamycin.

19. The method of any of claims 2-19, wherein the composition is administered in two or more doses.

20. The method of claim 20, wherein the interval of time between administration of doses of the composition is 0.5 to 30 days, 0.5 to 1 day, 1 to 3 days, 1 to 7 days, or 1-14 days.

21. The method of any of claim 2-3 or 6-21, wherein the composition is loaded into microparticles of a biodegradable polymer.

22. The method of claim 21, wherein the microparticles are disposed within an encasing material formulated for enteric release.

23. The method of claim 22, wherein the rapamycin or rapamycin analog is predominantly released in the colon.

24. The method of claim 22 or 23, wherein the biodegradable polymer comprises one or more of poly-ε-caprolactone, a polylactide, a polyglycolide, or combinations thereof.

25. The method of any of claims 22-24, wherein the encasing material comprises a pH-dependent polymer that dissolves in a pH-dependent manner.

26. The method of claim 25, wherein the pH-dependent polymer comprises a methyl methacrylate-methacrylic acid copolymer.

27. The method of claim 26, wherein the methyl methacrylate-methacrylic acid copolymer is Eudragit S 100.

28. The method of any of claims 22-28, wherein the encasing material comprises a hydrophilic gelling polymer or copolymer.

29. The method of claim 28, wherein the hydrophilic gelling polymer or copolymer comprises one or more of methylcellulose, carboxymethylcellulose, hydroxypropylcellulose, hydroxypropylmethylcellulose, carbomers, polyvinyl alcohols, polyoxyethylene glycols, polyvinylpyrrolidones, poloxamers, or natural or synthetic rubbers.

30. The method of any of claims 22-29, wherein the encasing material comprises one or more of chitosan, pectin, or a combination thereof.

31. The method of any of claims 22-30, wherein the encasing material comprises a starch capsule.

32. The method of claim 31, wherein the starch capsule comprises hydroxyethyl starch, hydroxypropyl starch, carboxymethyl starch, cationic starch, acetylated starch, phosphorylated starch, succinate derivatives, or grafted starches.

33. The method of any of claims 22-32, wherein the encasing material comprises a water-insoluble rupturable polymer layer.

34. The method of claim 33, wherein the rupturable polymer layer comprises cellulose acetate, cellulose acetate propionate, or ethyl cellulose.

35. The method of claim 33 or 34, wherein the rupturable polymer layer is semi-permeable.

36. The method of claim 35, wherein an effervescent material is disposed within the encasing material.

37. The method of any of claims 33-36, wherein the encasing material further comprises a swelling layer comprising croscarmellose sodium or hydroxyproplymethyl cellulose, and wherein the swelling layer is disposed within the rupturable polymer layer.

38. The method of claim 33 or 34, wherein a hydrophilic particulate material is embedded in the rupturable polymer layer, wherein the particulate material allows controlled entry of water past the rupturable polymer layer, wherein a swellable material is further disposed within the encasing material, and wherein the swellable material swells upon contact with water, causing the rupturable polymer layer to rupture.

39. The method of any of claims 22-38, wherein the encasing material comprises a wax matrix.

40. The method of claim 39, wherein the wax matrix comprises behenic acid.

41. The method of any of claims 22-40, wherein the encasing material comprises a first piece and a second piece, wherein the first piece contains an orifice, wherein the second piece is disposed initially to block the orifice and prevent entry of water, wherein the second piece comprises a swellable material, and wherein contacting the second piece with water causes it to swell and become displaced from the orifice.

42. The method of any of claims 22-41, wherein the composition is administered regularly for more than a week, more than a month, more than six months, more than one year, more than two years, more than three years, more than four years, or more than five years.

43. The method of any one of claims 1-42, wherein the individual is provided an effective amount of the composition no earlier than about 3 days following the procedure.

44. The method of any one of claims 1-42, wherein the individual is provided an effective amount of the composition no earlier than about 48 hours following the procedure.
